SEQUENCE LISTING (60 sequences)

SEQ ID NO: 1 - protein, 233 aa, synthetic construct
  REGION 1..233: note = B11
QEQLVESGGRLITPGGSLTLTCTVSGIDLNAYGVIWVRQAPGKGLEWIGGISRIGETWYASLVKGRFTISKTSTTVDLKMTSLTTEDTATYFCARERDAGAVGDGYTNDYFNIWGPGTLVTVSELVMTQTPASVSEPVGGTVTINCQASETIWSGLAWYQQKPGQPPKLLIYDASNLETGVPSRFRGSGSATQFTLTISDLECDDAATYYCQGPYYRSSGYFPFGGGTELEIL

SEQ ID NO: 2 - protein, 124 aa, synthetic construct
  REGION 1..124: note = B11 Heavy chain (VH)
QEQLVESGGRLITPGGSLTLTCTVSGIDLNAYGVIWVRQAPGKGLEWIGGISRIGETWYASLVKGRFTISKTSTTVDLKMTSLTTEDTATYFCARERDAGAVGDGYTNDYFNIWGPGTLVTVSL

SEQ ID NO: 3 - protein, 5 aa, synthetic construct
  REGION 1..5: note = B11 VH CDR1
AYGVI

SEQ ID NO: 4 - protein, 16 aa, synthetic construct
  REGION 1..16: note = B11 VH CDR2
GISRIGETWYASLVKG

SEQ ID NO: 5 - protein, 18 aa, synthetic construct
  REGION 1..18: note = B11 VH CDR3
ERDAGAVGDGYTNDYFNI

SEQ ID NO: 6 - protein, 110 aa, synthetic construct
  REGION 1..110: note = B11 Light chain (VL)
ELVMTQTPASVSEPVGGTVTINCQASETIWSGLAWYQQKPGQPPKLLIYDASNLETGVPSRFRGSGSATQFTLTISDLECDDAATYYCQGPYYRSSGYFPFGGGTELEIL

SEQ ID NO: 7 - protein, 11 aa, synthetic construct
  REGION 1..11: note = B11 VL CDR1
QASETIWSGLA

SEQ ID NO: 8 - protein, 7 aa, synthetic construct
  REGION 1..7: note = B11 VL CDR2
DASNLET

SEQ ID NO: 9 - protein, 12 aa, synthetic construct
  REGION 1..12: note = B11 VL CDR3
QGPYYRSSGYFP

SEQ ID NO: 10 - protein, 218 aa, synthetic construct
  REGION 1..218: note = E6
QSVEESGGRLVTPGTPLTLTCTVSGFSLSSYPVSWVRQAPGKGLEWIGFINTGGSAYYASWAKGRFTISRTSTSVDLKMASLTASDTATYFCGVNGIWGPGTLVTVSLELDMTQTPPSLSASVGETVRITCLASEDIYSGISWYQQKPGKPPTLLISGASNLESGVPPRFSGSGSGTDYTLTIGGVQAEDAATYYCLGGVSFSTTGTTFGAGTNVEIK

SEQ ID NO: 11 - protein, 108 aa, synthetic construct
  REGION 1..108: note = E6 Heavy chain (VH)
QSVEESGGRLVTPGTPLTLTCTVSGFSLSSYPVSWVRQAPGKGLEWIGFINTGGSAYYASWAKGRFTISRTSTSVDLKMASLTASDTATYFCGVNGIWGPGTLVTVSL

SEQ ID NO: 12 - protein, 5 aa, synthetic construct
  REGION 1..5: note = E6 VH CDR1
SYPVS

SEQ ID NO: 13 - protein, 16 aa, synthetic construct
  REGION 1..16: note = E6 VH CDR2
FINTGGSAYYASWAKG

SEQ ID NO: 14 - skipped

SEQ ID NO: 15 - protein, 110 aa, synthetic construct
  REGION 1..110: note = E6 Light Chain (VL)
ELDMTQTPPSLSASVGETVRITCLASEDIYSGISWYQQKPGKPPTLLISGASNLESGVPPRFSGSGSGTDYTLTIGGVQAEDAATYYCLGGVSFSTTGTTFGAGTNVEIK

SEQ ID NO: 16 - protein, 11 aa, synthetic construct
  REGION 1..11: note = E6 VL CDR1
LASEDIYSGIS

SEQ ID NO: 17 - protein, 7 aa, synthetic construct
  REGION 1..7: note = E6 VL CDR2
GASNLES

SEQ ID NO: 18 - protein, 12 aa, synthetic construct
  REGION 1..12: note = E6 VL CDR3
LGGVSFSTTGTT

SEQ ID NO: 19 - protein, 233 aa, synthetic construct
  REGION 1..233: note = G4
QSLEESGGRLVTPGGSLTLTCTVSGIDLNSYGVIWVRQAPGKGLEWIGGISRIGETWYASLVKGRFTISKTSTTVDLKMTSLTTEDTATYFCARERDAGAVGDGYTNDYFNIWGPGTLVIVSLELVMTQTPASVSEPVGGTVTINCQASETIWSGLAWYQQKPGQPPKLLIYDASNLETGVPSRFRGSGSATQFTLTISDLECDDAATYYCQGPYYRSSGYFPFGGGTELEIL

SEQ ID NO: 20 - protein, 123 aa, synthetic construct
  REGION 1..123: note = G4 Heavy chain (VH)
QSLEESGGRLVTPGGSLTLTCTVSGIDLNSYGVIWVRQAPGKGLEWIGGISRIGETWYASLVKGRFTISKTSTTVDLKMTSLTTEDTATYFCARERDAGAVGDGYTNDYFNIWGPGTLVIVSL

SEQ ID NO: 21 - protein, 5 aa, synthetic construct
  REGION 1..5: note = G4 VH CDR1
SYGVI

SEQ ID NO: 22 - protein, 16 aa, synthetic construct
  REGION 1..16: note = G4 VH CDR2
GISRIGETWYASLVKG

SEQ ID NO: 23 - protein, 18 aa, synthetic construct
  REGION 1..18: note = G4 VH CDR3
ERDAGAVGDGYTNDYFNI

SEQ ID NO: 24 - protein, 110 aa, synthetic construct; identical to SEQ ID NO: 6
  REGION 1..110: note = G4 Light chain (VL)

SEQ ID NO: 25 - protein, 11 aa, synthetic construct
  REGION 1..11: note = G4 VL CDR1
QASETIWSGLA

SEQ ID NO: 26 - protein, 7 aa, synthetic construct
  REGION 1..7: note = G4 VL CDR2
DASNLET

SEQ ID NO: 27 - protein, 12 aa, synthetic construct
  REGION 1..12: note = G4 VL CDR3
QGPYYRSSGYFP

SEQ ID NO: 28 - DNA, 702 bp, synthetic construct
  misc_feature 1..702: note = B11
  source 1..702: mol_type = other DNA
caggagcaattggtggaatcaggcgggagactgattaccccaggtggaagccttacactgacttgcactgtctcagggattgacctgaatgcctatggcgtcatctgggttcgccaagcaccagggaaagggctggagtggattggaggcataagtcggattggggaaacctggtatgccagtctggtgaaaggtcgctttactatctccaagacatccaccacagtggacctcaagatgacctctctgactacggaggacacagctacctatttctgtgcacgagagagggatgctggagctgttggtgatgggtacaccaacgactacttcaacatctggggacccggcactcttgtgaccgtgagcctggagctggtcatgacacagacccctgctagcgtgtcagaaccagtgggtggtacagtcaccatcaattgccaggcttccgagacaatatggtccggattggcctggtatcagcagaagcctggacagccacccaaactcctgatctacgatgcctccaacctggaaacaggcgtacctagccgttttcgaggcagtggctctgcaactcagttcacccttacgatcagcgacttggaatgcgatgatgccgcaacctactactgtcaagggccctattatcggtctagtggatactttccctttggcggcggcactgagctggagattctc

SEQ ID NO: 29 - DNA, 372 bp, synthetic construct
  misc_feature 1..372: note = B11 Heavy chain (VH)
  source 1..372: mol_type = other DNA
caggagcaattggtggaatcaggcgggagactgattaccccaggtggaagccttacactgacttgcactgtctcagggattgacctgaatgcctatggcgtcatctgggttcgccaagcaccagggaaagggctggagtggattggaggcataagtcggattggggaaacctggtatgccagtctggtgaaaggtcgctttactatctccaagacatccaccacagtggacctcaagatgacctctctgactacggaggacacagctacctatttctgtgcacgagagagggatgctggagctgttggtgatgggtacaccaacgactacttcaacatctggggacccggcactcttgtgaccgtgagcctg

SEQ ID NO: 30 - DNA, 330 bp, synthetic construct
  misc_feature 1..330: note = B11 light chain (VL)
  source 1..330: mol_type = other DNA
gagctggtcatgacacagacccctgctagcgtgtcagaaccagtgggtggtacagtcaccatcaattgccaggcttccgagacaatatggtccggattggcctggtatcagcagaagcctggacagccacccaaactcctgatctacgatgcctccaacctggaaacaggcgtacctagccgttttcgaggcagtggctctgcaactcagttcacccttacgatcagcgacttggaatgcgatgatgccgcaacctactactgtcaagggccctattatcggtctagtggatactttccctttggcggcggcactgagctggagattctc

SEQ ID NO: 31 - DNA, 654 bp, synthetic construct
  misc_feature 1..654: note = E6
  source 1..654: mol_type = other DNA
cagtcggtggaggagtccgggggtcgcctggtcacgcctgggacacccctgacactcacctgcacagtctctggattctccctcagtagctatccagtgagctgggtccgccaggctccagggaaggggctggaatggatcggatttattaatactggtggtagcgcatactacgcgagctgggcaaaaggccgattcaccatctccagaacctcgacctcggtggatctgaaaatggccagtctgacagcctcggacacggccacctatttctgtggtgtaaatggtatctggggcccaggcaccctggtcaccgtctccttagagctcgatatgacccagactccaccctccctgtctgcatctgtgggagaaactgtcaggattacgtgcctggccagtgaggacatttacagtggtatatcctggtatcaacagaagccagggaaacctcctacactcctgatctctggtgcatccaatttagaatctggggtcccaccacggttcagtggcagtggatctgggacagattacaccctcaccattggcggcgtgcaggctgaagatgctgccacctactactgtctaggcggtgttagtttcagtactaccggtacgacttttggagctggcaccaatgtggaaatcaaa

SEQ ID NO: 32 - DNA, 324 bp, synthetic construct
  misc_feature 1..324: note = E6 Heavy chain (VH)
  source 1..324: mol_type = other DNA
cagtcggtggaggagtccgggggtcgcctggtcacgcctgggacacccctgacactcacctgcacagtctctggattctccctcagtagctatccagtgagctgggtccgccaggctccagggaaggggctggaatggatcggatttattaatactggtggtagcgcatactacgcgagctgggcaaaaggccgattcaccatctccagaacctcgacctcggtggatctgaaaatggccagtctgacagcctcggacacggccacctatttctgtggtgtaaatggtatctggggcccaggcaccctggtcaccgtctcctta

SEQ ID NO: 33 - DNA, 331 bp, synthetic construct
  misc_feature 1..331: note = E6 Light chain (VL)
  source 1..331: mol_type = other DNA
gagctcgatatgacccagactccaccctccctgtctgcatctgtgggagaaactgtcaggattacgtgcctggccagtgaggacatttacagtggtatatcctggtatcaacagaagccagggaaacctcctacactcctgatctctggtgcatccaatttagaatctggggtcccaccacggttcagtggcagtggatctgggacagattacaccctcaccattggcggcgtgcaggctgaagatgctgccacctactactgtctaggcggtgttagtttcagtactaccggtacgacttttggagctggcaccaatgtggaaatcaaac

SEQ ID NO: 34 - DNA, 699 bp, synthetic construct
  misc_feature 1..699: note = G4
  source 1..699: mol_type = other DNA
cagtcgctggaggagtccgggggtcgcctggtaacgcctggaggatccctgacactcacctgcacagtctctggaatcgacctcaattcttatggagtgatctgggtccgccaggctccagggaaggggctggaatggatcggaggcatcagtaggattggtgagacatggtacgcgagcttggtgaaaggccgattcaccatctccaaaacctcgaccacggtggatctgaaaatgaccagtctgacaaccgaggacacggccacctatttctgtgccagagaacgtgatgctggtgcggttggtgatggttatactaacgactactttaatatctggggcccaggcaccctggtcatcgtctccttagagctcgtgatgacccagactccagcctccgtgtctgaacctgtgggaggcacagtcaccatcaattgccaggccagtgagactatttggagtggtttggcctggtatcagcagaaaccagggcagcctcccaaactcctgatctatgatgcatccaatctggagactggggtcccatcgcggttcagaggcagtggatctgcgacacagttcactctcaccatcagcgacctggagtgtgacgatgctgccacttactattgtcaaggtccttattataggagtagtggctattttcctttcggcggagggaccgagctggagatccta

SEQ ID NO: 35 - DNA, 369 bp, synthetic construct
  misc_feature 1..369: note = G4 Heavy Chain (VH)
  source 1..369: mol_type = other DNA
cagtcgctggaggagtccgggggtcgcctggtaacgcctggaggatccctgacactcacctgcacagtctctggaatcgacctcaattcttatggagtgatctgggtccgccaggctccagggaaggggctggaatggatcggaggcatcagtaggattggtgagacatggtacgcgagcttggtgaaaggccgattcaccatctccaaaacctcgaccacggtggatctgaaaatgaccagtctgacaaccgaggacacggccacctatttctgtgccagagaacgtgatgctggtgcggttggtgatggttatactaacgactactttaatatctggggcccaggcaccctggtcatcgtctcctta

SEQ ID NO: 36 - DNA, 330 bp, synthetic construct
  misc_feature 1..330: note = G4 Light chain (VL)
  source 1..330: mol_type = other DNA
gagctcgtgatgacccagactccagcctccgtgtctgaacctgtgggaggcacagtcaccatcaattgccaggccagtgagactatttggagtggtttggcctggtatcagcagaaaccagggcagcctcccaaactcctgatctatgatgcatccaatctggagactggggtcccatcgcggttcagaggcagtggatctgcgacacagttcactctcaccatcagcgacctggagtgtgacgatgctgccacttactattgtcaaggtccttattataggagtagtggctattttcctttcggcggagggaccgagctggagatccta

SEQ ID NO: 37 - protein, 30 aa, synthetic construct
  REGION 1..30: note = B11 HFR1
QEQLVESGGRLITPGGSLTLTCTVSGIDLN

SEQ ID NO: 38 - protein, 14 aa, synthetic construct
  REGION 1..14: note = B11 HFR2
WVRQAPGKGLEWIG

SEQ ID NO: 39 - protein, 30 aa, synthetic construct
  REGION 1..30: note = B11 HFR3
RFTISKTSTTVDLKMTSLTTEDTATYFCAR

SEQ ID NO: 40 - protein, 11 aa, synthetic construct
  REGION 1..11: note = B11 HFR4
WGPGTLVTVSL

SEQ ID NO: 41 - protein, 23 aa, synthetic construct
  REGION 1..23: note = B11 LFR1
ELVMTQTPASVSEPVGGTVTINC

SEQ ID NO: 42 - protein, 15 aa, synthetic construct
  REGION 1..15: note = B11 LFR2
WYQQKPGQPPKLLIY

SEQ ID NO: 43 - protein, 32 aa, synthetic construct
  REGION 1..32: note = B11 LFR3
GVPSRFRGSGSATQFTLTISDLECDDAATYYC

SEQ ID NO: 44 - protein, 10 aa, synthetic construct
  REGION 1..10: note = B11 LFR4
FGGGTELEIL

SEQ ID NO: 45 - protein, 29 aa, synthetic construct
  REGION 1..29: note = E6 HFR1
QSVEESGGRLVTPGTPLTLTCTVSGFSLS

SEQ ID NO: 46 - protein, 14 aa, synthetic construct
  REGION 1..14: note = E6 HFR2
WVRQAPGKGLEWIG

SEQ ID NO: 47 - protein, 30 aa, synthetic construct
  REGION 1..30: note = E6 HFR3
RFTISRTSTSVDLKMASLTASDTATYFCGV

SEQ ID NO: 48 - protein, 11 aa, synthetic construct
  REGION 1..11: note = E6 HFR4
WGPGTLVTVSL

SEQ ID NO: 49 - protein, 23 aa, synthetic construct
  REGION 1..23: note = E6 LFR1
ELDMTQTPPSLSASVGETVRITC

SEQ ID NO: 50 - protein, 15 aa, synthetic construct
  REGION 1..15: note = E6 LFR2
WYQQKPGKPPTLLIS

SEQ ID NO: 51 - protein, 32 aa, synthetic construct
  REGION 1..32: note = E6 LFR3
GVPPRFSGSGSGTDYTLTIGGVQAEDAATYYC

SEQ ID NO: 52 - protein, 10 aa, synthetic construct
  REGION 1..10: note = E6 LFR4
FGAGTNVEIK

SEQ ID NO: 53 - protein, 29 aa, synthetic construct
  REGION 1..29: note = G4 HFR1
QSLEESGGRLVTPGGSLTLTCTVSGIDLN

SEQ ID NO: 54 - protein, 14 aa, synthetic construct
  REGION 1..14: note = G4 HFR2
WVRQAPGKGLEWIG

SEQ ID NO: 55 - protein, 30 aa, synthetic construct; identical to SEQ ID NO: 39
  REGION 1..30: note = G4 HFR3

SEQ ID NO: 56 - protein, 11 aa, synthetic construct
  REGION 1..11: note = G4 HFR4
WGPGTLVIVSL

SEQ ID NO: 57 - protein, 23 aa, synthetic construct
  REGION 1..23: note = G4 LFR1
ELVMTQTPASVSEPVGGTVTINC

SEQ ID NO: 58 - protein, 15 aa, synthetic construct
  REGION 1..15: note = G4 LFR2
WYQQKPGQPPKLLIY

SEQ ID NO: 59 - protein, 32 aa, synthetic construct; identical to SEQ ID NO: 43
  REGION 1..32: note = G4 LFR3

SEQ ID NO: 60 - protein, 10 aa, synthetic construct
  REGION 1..10: note = G4 LFR4
FGGGTELEIL

(19) United States
(12) Patent Application Publication
(10) Pub. No.: US 2024/0385186 A1
(43) Pub. Date: Nov. 21, 2024

(54) ANTIBODY AND ANTIGEN BINDING FRAGMENTS THEREOF

(71) Applicant: Philip Morris Products S.A., Neuchatel (CH)

(72) Inventors: Laura FRANCIUS, Guipry (FR); Nikolai IVANOV, Neuchatel (CH); Maxime LAROCHE, Guipry (FR); Thibaut PELAT, Apprieu (FR); Marja TALIKKA, Neuchatel (CH)

(73) Assignee: Philip Morris Products S.A., Neuchatel (CH)

(21) Appl. No.: 18/683,643

(22) PCT Filed: Aug. 15, 2022
(86) PCT No.: PCT/EP2022/072779

§ 371 Date: Feb. 14, 2024

(30) Foreign Application Priority Data
Aug. 18, 2021 (EP) 21191986.5

(51) Int. Cl. G01N 33/543; C07K 16/44; G01N 33/58; G01N 33/94

(52) U.S. Cl. CPC G01N 33/54388; C07K 16/44; G01N 33/58; G01N 33/94; C07K 2317/565; C07K 2317/622

(57) ABSTRACT

There is disclosed an antibody or antigen binding fragment thereof: (i) capable of binding to N-acetyl-S—[2-carboxyethyl]-L-cysteine (CEMA) and; (ii) capable of binding to a conjugate comprising a compound of formula [II] wherein n is selected from 0 to 4 (that is, 0, 1, 2, 3, or 4), and each R is independently selected from H or C₁ to C₆ alkyl; preferably, a compound of formula [I] wherein the compound of formula [II] or formula [I] is coupled to an immunogenic carrier via a linker, suitably, wherein the linker is coupled to the compound of formula [I] via the amine group.

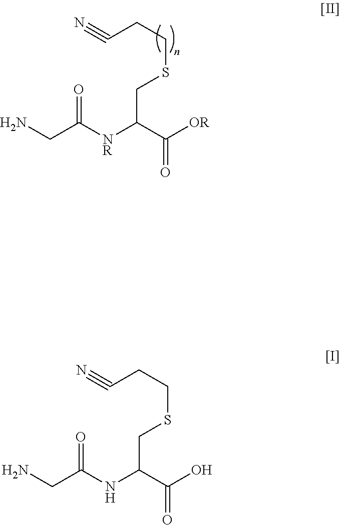

### CROSS-REFERENCE TO RELATED APPLICATIONS

This application is a national-stage entry of PCT/EP20221072779, filed Aug. 15, 2022 which claims priority to EPC 21191986.5, filed Aug. 18, 2021. Both of these applications are incorporated by reference for all purposes.

### REFERENCE TO A SEQUENCE LISTING

In accordance with 37 CFR § 1.833-1835 and 37 CFR § 1.77(b)(5), the specification makes reference to a Sequence Listing submitted electronically as an .xml file named “550536US_ST26.xml”. The .xml file was generated on Jan. 12, 2024 and is 79,786 bytes in size. The entire contents of the Sequence Listing are hereby incorporated by reference.

### FIELD OF THE INVENTION

The present invention relates, in general, to novel antibodies or antigen binding fragments thereof that can be used in immunoassays to assist in determining or distinguishing the smoking status of a subject.

### BACKGROUND

Aerosol generating articles in which tobacco is heated rather than combusted have been proposed in the art. In heated aerosol generating articles, an aerosol is generated by heating a substrate—such as tobacco. Studies have shown that heating tobacco to temperatures below pyrolysis and combustion temperatures have the potential to reduce or eliminate some toxicants found in cigarette smoke. Heating instead of burning tobacco, typically at temperatures lower than 300° C. is sufficient to release nicotine, but not high enough to cause significant pyrolysis. At these temperatures, the aerosol composition becomes simpler than that found in cigarette smoke. Many ‘harmful and potentially harmful constituents’ (HPHCs) in cigarette smoke are formed due to the combustion of tobacco. Thus, lowering the temperature and heating the tobacco instead of burning it can reduce or eliminate HPHCs. Known heated aerosol generating articles include electrically heated aerosol generating articles and aerosol generating articles in which an aerosol is generated by the transfer of heat from a combustible fuel element or heat source to a physically separate aerosol forming material. These so called ‘heat-not-burn’ products offers smokers alternatives to conventional cigarettes and may reduce harmful chemicals released from the tobacco while still delivering nicotine. One such tobacco heating system is IQOS (THS), which contains sophisticated electronics to heat specially designed heated tobacco units. THS heats the tobacco just enough to release a nicotine-containing tobacco vapor but without burning the tobacco. The tobacco in a cigarette burns at temperatures in excess of 600° C., generating smoke that contains high levels of harmful chemicals. But THS heats tobacco to much lower temperatures, up to 350° C., without combustion, fire, ash, or smoke. Because the tobacco is heated and not burned, the levels of harmful chemicals are significantly reduced compared to cigarette smoke.

A diagnostic test to determine or distinguish the smoking status of a subject has been described in WO2018/211126. By way of example, this test can distinguish between current smokers of conventional cigarettes (‘smoker’), those who have switched to a ‘heat-not-burn’ product, also known as Reduced Risk Product or RRP (‘switcher’) or those who have abstained from smoking (‘non-smoker’). Such a test can have a variety of applications. For example, the test can be used in clinical trials for identifying and screening of subjects based on their smoking status. By way of further example, the test can be used for insurance purposes as a compliance test to monitor switching to a RRP and compliance with the switch. Various metabolites are described in WO2018/211126 that can be used as tobacco smoke exposure biomarkers, including Cotinine and cyanoethyl mercapturic acid (CEMA). There is a need in the art for agents that can detect CEMA with high specificity and sensitivity, particularly for use in immunoassay format. Such agents should be easy to manufacture, have good solubility and diffusion and be able to detect CEMA specifically and sensitively in complex biological samples—such as urine. The present invention seeks to address this need.

### SUMMARY OF THE INVENTION

The present invention is based, at least in part, on the surprising finding that a novel conjugate comprising a compound of formula [II]:

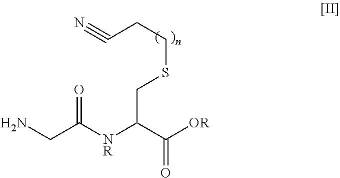

wherein n is selected from 0 to 4 (that is, 0, 1, 2, 3, or 4), and each R is independently selected from H or C₁ to C₆ alkyl, coupled to an immunogenic carrier via a linker, is an excellent conjugate to elicit a robust immune response to obtain anti-CEMA antibodies or antigen binding fragments thereof. Conjugation of CEMA to the immunogenic carrier via a linker improves recognition by the immune system and the production of antibodies. Often, this so called ‘anti-hapten antibody production’ can result in reagents with limited target affinity. Advantageously however, the antibody or antigen binding fragments of the present disclosure—particularly in scFv format—are highly sensitive and specific for CEMA even in complex biological samples including urine. Out of 96 reactive clones that were obtained during phage display screening, 28 positive clones were identified. From these 28 positive clones, 8 sequences were harvested to produce 8 scFv which were tested in immunoassays. Of these 8 scFv, 3 were selected based on their excellent sensitivity and specificity for CEMA and are referred to herein as ‘scFv G4’, ‘scFv B11’ and ‘scFv E6’, or ‘G4’, ‘B11’ and ‘E6’.

### ASPECTS AND EMBODIMENTS OF THE INVENTION

In one aspect, there is disclosed an antibody or antigen binding fragment thereof: (i) capable of binding to N-acetyl-S-[2-carboxyethyl]-L-cysteine (CEMA) and; (ii) capable of binding to a conjugate comprising a compound of formula [II]:

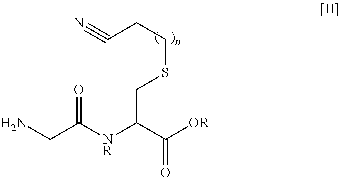

wherein n is selected from 0 to 4 (that is, 0, 1, 2, 3, or 4), and each R is independently selected from H or C₁ to C₆ alkyl; preferably, a compound of formula [I]:

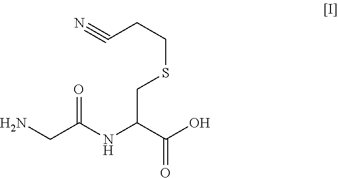 [SMILES: N#CCCSCC(NC(=O)CN)C(=O)O]

wherein the compound of formula [II] or formula [I] is coupled to an immunogenic carrier via a linker, suitably, wherein the linker is coupled to the compound of formula [I] via the amine group.

Suitably, the immunogenic carrier is a protein, preferably, bovine serum albumin or bovine thyroglobulin.

Suitably, the linker is glycol bis(succinimidyl succinate) (EGS) or disuccinimidyl suberate (DSS).

Suitably, the antibody is a monoclonal antibody, preferably, wherein the antigen binding fragment thereof is a Fab fragment, a Fab′ fragment, a F(ab′)2 fragment, a scFv, a Fv, a rIgG, or a diabody, more preferably a scFv.

Suitably, the antibody or antigen binding fragment thereof does not bind to Cotinine or 2-hydroxyethylmethacrylate (HEMA) or monohydroxybutenyl-mercapturic acid (MHBMA) or 3-hydroxypropylmercapturic acid (3-HPMA) or dihydroxybutyl mercapturic acid (DHBMA).

Suitably, the percentage inhibition by urine is 20% or less, 10% or less, 5% or less or no inhibition.

Suitably, the antibody or antigen binding fragment thereof has a limit of detection for CEMA of 160 ng/mL CEMA in urine in an immunoassay.

In another aspect, there is disclosed an antibody or antigen binding fragment thereof comprising: VH CDR1, VH CDR2 and VH CDR3 consisting of the amino acid sequences of SEQ ID NO:3, SEQ ID NO:4 and SEQ ID NO:5 respectively, and VL CDR1, VL CDR2 and VL CDR3 consisting of the amino acid sequences of SEQ ID NO:7, SEQ ID NO:8 and SEQ ID NO:9 respectively; or VH CDR1, VH CDR2 and VH CDR3 consisting of the amino acid sequences of SEQ ID NO:12, SEQ ID NO:13 and SEQ ID NO:14 respectively, and VL CDR1, VL CDR2 and VL CDR3 consisting of the amino acid sequences of SEQ ID NO:16, SEQ ID NO:17 and SEQ ID NO:18 respectively; or VH CDR1, VH CDR2 and VH CDR3 consisting of the amino acid sequences of SEQ ID NO:21, SEQ ID NO:22 and SEQ ID NO:23 respectively, and VL CDR1, VL CDR2 and VL CDR3 consisting of the amino acid sequences of SEQ ID NO:25, SEQ ID NO:26 and SEQ ID NO:27 respectively.

Suitably, the antibody or antigen binding fragment thereof comprises VH amino acid sequence consisting of the amino acid sequence of SEQ ID NO: 2 or 11 or 20.

Suitably, the antibody or antigen binding fragment thereof comprises VL amino acid sequence consisting of the amino acid sequence of SEQ ID NO:6 or 15 or 24.

Suitably, the antibody or antigen binding fragment thereof comprises VH amino acid sequence consisting of the amino acid sequence of SEQ ID NO: 2 and VL amino acid sequence consisting of the amino acid sequence of SEQ ID NO:6; or

Suitably, the antibody or antigen binding fragment thereof comprises VH amino acid sequence consisting of the amino acid sequence of SEQ ID NO:11 and VL amino acid sequence consisting of the amino acid sequence of SEQ ID NO:15; or wherein said antibody or antigen binding fragment thereof comprises VH amino acid sequence consisting of the amino acid sequence of SEQ ID NO:20 and VL amino acid sequence consisting of the amino acid sequence of SEQ ID NO:24.

Suitably, the antigen binding fragment thereof is selected from the group consisting of a Fab fragment, a Fab′ fragment, a F(ab′)₂ fragment, a scFv, a Fv, a rIgG, and a diabody, preferably scFv.

Suitably, the antigen binding fragment is a scFv and wherein said scFv comprises the amino acid sequence of SEQ ID NO: 1 or SEQ ID NO: 10 or SEQ ID NO: 19.

In another aspect, there is disclosed a polynucleotide encoding the antibody or antigen binding fragment thereof as described herein or a polynucleotide complementary thereto.

Suitably, the polynucleotide comprises one or more polynucleotide sequences selected from the group consisting of SEQ ID NO:28, SEQ ID NO: 31 and SEQ ID NO:34 or a polynucleotide complementary thereto.

In a further aspect, there is provided a vector comprising a polynucleotide sequence.

Suitably, the vector further comprises an expression control sequence operatively linked to the nucleic acid encoding the variable heavy chain domain and/or the variable light chain domain.

In a further aspect, there is provided a host cell containing the vector.

Suitably, the host cell is a eukaryotic cell or a prokaryotic cell.

Suitably, the eukaryotic cell is a Chinese Hamster Ovary (CHO) cell.

Suitably, the prokaryotic cell is an E. coli cell.

In a further aspect, there is provided a method of producing an antibody or antigen binding fragment thereof, comprising incubating the host cell such that the encoded variable heavy chain domain and/or variable light chain domain is expressed by the cell; and recovering the expressed antibody or antigen binding fragment thereof.

Suitably, the method further comprises isolating and/or purifying the recovered antibody or antigen binding fragment thereof.

In another aspect, there is disclosed a method for producing the antibody described herein, comprising immunising a non-human animal with a conjugate comprising a compound of formula [II]:

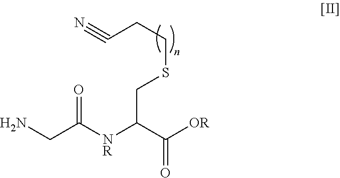

wherein n is selected from 0 to 4 (that is, 0, 1, 2, 3, or 4), and each R is independently selected from H or C₁ to C₆ alkyl; preferably, a compound of formula [I]:

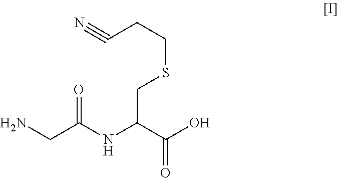 [SMILES: N#CCCSCC(NC(=O)CN)C(=O)O]

wherein said compound of formula [II] or formula [I] is coupled to an immunogenic carrier via a linker, suitably, wherein the linker is coupled to the compound of formula [I] via the amine group.

In another aspect, there is disclosed a device for determining the presence or absence of CEMA in a sample, said device comprising the antibody or antigen binding fragment thereof described herein immobilised on a solid phase of the device.

In another aspect, there is disclosed a device for determining the presence or absence of CEMA in a sample, said device comprising the conjugate described herein immobilised on a solid phase of the device.

Suitably, the device is a portable lateral flow immunoassay device, preferably, a dipstick.

Suitably, the device comprises: (i) a sample pad for receiving a sample; (ii) a conjugate pad in fluid communication with the sample pad; (iii) at least one detection zone in fluid communication with a distal end of the conjugate pad; and (iv) an adsorbent pad in fluid communication with a distal end of the detection zone.

Suitably, the conjugate pad comprises the antibody or antigen binding fragment thereof according to the present invention and as described above, wherein said antibody or antigen binding fragment thereof is labelled; and optionally a labelled antibody or antigen binding fragment thereof capable of binding cotinine Suitably, the antibody or antigen binding fragment thereof according to the present invention and described above is labelled and is separately contained in an intermediate pad of the device that is located adjacent the conjugate pad and optionally, wherein, a labelled antibody or antigen binding fragment thereof capable of binding Cotinine is separately contained in the conjugate pad. Suitably, the detection zone comprises CEMA and optionally Cotinine immobilised thereon, suitably, wherein the CEMA is in the form of the conjugate of the present invention and as described above.

In another aspect, there is disclosed a method for detecting CEMA in a sample comprising the use of the device described herein or a method for detecting CEMA and Cotinine in a sample comprising the use of the device described herein.

Suitably, the method comprises: (i) applying an aliquot of a liquid biological sample, preferably urine, to the sample pad, whereby the liquid biological sample is transferred by capillary action along a flow path defined by the sample pad, the conjugate pad, the detection zone; and the adsorbent pad; and (ii) determining the presence or absence of CEMA in the detection zone and optionally determining the presence or absence of Cotinine in the detection zone.

In another aspect, there is disclosed the use of the antibody or antigen binding fragment thereof as described herein or the device as described herein for detecting CEMA in a sample.

### BRIEF DESCRIPTION OF THE DRAWINGS

FIG. 1 illustrates ELISA assay results in the form of graphs showing reactivity of different scFv clones on different immunogens and proteins, including scFv G4, scFv B11 and scFv E6. The y-axis denotes the optical density at 450 nm and the x-axis denotes the dilution of CEMA. The optical density is measured in a BioteK EL808 reader as described in the accompanying Examples.

FIG. 2 presents a table and a graph of scFv competitive ELISA results using the BTG-EGS-CEMA immunogen and free CEMA. For each data set, the columns represent from left to right results at 10, 1, 0.1, 0.01, 0.001 and 0.0001 μg/ml free CEMA. The y-axis denotes the optical density at 450 nm and the x-axis denotes the scFv conjugate. The optical density is measured in a BioteK EL808 reader as described in the accompanying Examples.

FIG. 3 presents a table and a graph of scFv competitive ELISA results using the BTG-DSS-CEMA immunogen and free CEMA. For each data set, the columns represent from left to right results at 10 μg/ml, 1 μg/ml, 0.1 μg/ml, 0.01 μg/ml, 0.001 μg/ml and 0.0001 μg/ml free CEMA. The y-axis denotes the optical density at 450 nm and the x-axis denotes the scFv conjugate. The optical density is measured in a BioteK EL808 reader as described in the accompanying Examples.

FIG. 4 presents a graph showing lateral flow immunoassay reactivity testing results in which the reactivity of selected scFVs are tested against the BTG-EGS-CEMA immunogen and the BTG-DSS-CEMA immunogen. For each data set, the columns represent from left to right results with BTG-EGS-CEMA immunogen or BTG-DSS-CEMA immunogen. The y-axis denotes the signal intensity measured in the lateral flow immunoassay and the x-axis denotes the scFv conjugate under test.

FIG. 5 presents a graph showing lateral flow immunoassay test results investigating scFV inhibition with free CEMA and the BTG-EGS-CEMA immunogen. For each data set, the columns represent from left to right results with 0 μg/ml (PBS 1×), negative urine, CEMA 500 ng/ml (urine), CEMA 100 ng/ml (urine), CEMA 50 ng/ml (urine), CEMA 20 ng/ml (urine) and CEMA 10 ng/ml (urine). The y-axis denotes the signal intensity measured in the lateral flow immunoassay and the x-axis denotes the scFv conjugate under test.

FIG. 6 presents a graph showing lateral flow immunoassay test results investigating scFV inhibition with free CEMA and the BTG-DSS-CEMA immunogen. For each data set, the columns represent from left to right results with 0 μg/ml (PBS 1×), negative urine, CEMA 500 ng/ml (urine), CEMA 100 ng/ml (urine), CEMA 50 ng/ml (urine), CEMA 20 ng/ml (urine) and CEMA 10 ng/ml (urine). The y-axis denotes the signal intensity measured in the lateral flow immunoassay and the x-axis denotes the scFv conjugate under test.

FIG. 7 presents a graph showing lateral flow immunoassay test results of the inhibition profile of selected scFv in urine samples. For each data set, the columns represent from left to right results with G4, B11 or E6 scFv. The y-axis denotes the signal intensity measured in the lateral flow immunoassay and the x-axis denotes the CEMA amount under test.

FIG. 8 presents a graph showing specificity of scFV G4, scFV B11 and scFV E6 against Cotinine and molecules with a structure close to CEMA, which are 2-hydroxyethyl mercapturic acid (HEMA), monohydroxybutenylmercapturic acid (MHBMA), 3-hydroxypropyl mercapturic acid (3-HPMA) and dihydroxybutylmercapturic acid (DHBMA), as measured in a measured in a lateral flow immunoassay. For each data set, the columns represent from left to right results with G4, B11 or E6 scFv. The y-axis denotes the signal intensity measured in the lateral flow immunoassay and the x-axis denotes the molecule under test for interference.

FIG. 9 is a drawing illustrating a portable lateral flow immunoassay device format according to an aspect of the present disclosure.

FIG. 10 is a drawing illustrating multiplex test protocols that can be obtained on a portable lateral flow immunoassay device and their interpretation. C=Control; CEM=CEMA test line; COT=Cotinine test line.

### DEFINITIONS

Section headings as used in this disclosure are for organisation purposes and are not intended to be limiting. Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. The materials, methods, and examples disclosed herein are illustrative only and not intended to be limiting.

As used herein, the singular forms “a”, “an”, and “the” include both singular and plural referents unless the context clearly dictates otherwise.

The term “and/or” means (a) or (b) or both (a) and (b).

The terms “comprising”, “comprises” and “comprised of” as used herein are synonymous with “including”, “includes” or “containing”, “contains”, and are inclusive or open-ended and do not exclude additional, non-recited members, elements or method steps.

The term “consisting of” means that additional components are excluded and has the recited elements only and no more.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosure. It is to be understood that the value to which the modifier “about” refers is itself also specifically, and preferably, disclosed.

As used herein, the term “antibody” encompasses intact monoclonal antibodies, polyclonal antibodies, multivalent (for example, 2-, 3- or more-valent) and/or multi-specific antibodies (for example, bi- or more-specific antibodies) formed from at least two intact antibodies, and antigen-binding fragments thereof insofar they exhibit the desired biological activity (particularly, ability to specifically bind metabolite—such as CEMA), as well as multivalent and/or multi-specific composites of such fragments. The term is not only inclusive of antibodies generated by methods comprising immunisation, but also includes any polypeptide, for example, a recombinantly expressed or synthetic polypeptide, which is made to encompass at least one complementarity-determining region (CDR) capable of specifically binding to an epitope. Hence, the term applies to such molecules regardless whether they are produced in vitro or in vivo.

The term ‘isolated’ refers to the removal of a molecule from its natural environment.

Whereas the term “one or more”, such as one or more members of a group of members, is clear per se, by means of further exemplification, the term encompasses inter alia a reference to any one of said members, or to any two or more of said members, such as—any ≥3, ≥4, ≥5, ≥6 or ≥7 etc. of said members, and up to all said members.

The term “label” refers to any atom, molecule, moiety or biomolecule that can be used to provide a detectable and preferably quantifiable read-out or property, and that may be attached to or made part of an entity of interest, such as a metabolite or an antibody or antigen-binding fragment thereof.

The term “metabolite” is widespread in the art and may broadly denote any substance produced by metabolism or by a metabolic process. Expressed in another way, a metabolite is an end product resulting from metabolism. The term also encompasses a detectable portion of a metabolite whose qualitative and/or quantitative evaluation in a subject is, alone or combined with other data, informative with respect to the status of the subject as to switching compliance. The metabolites are typically small molecules derived from combustible tobacco products. Monitoring of the metabolites over time may enable the progress of a subject's switching compliance to be determined over time.

A molecule is “measured” in a sample when the presence or absence and/or quantity of said molecule or of said group of molecules is detected or determined in the sample, preferably substantially to the exclusion of other molecules. For example, a molecule may be measured by laboratory tests as described herein.

The term “non-smoker” means a subject that has previously been a smoker but has not smoked tobacco products (for example, cigarettes) in the past 3 months. The non-smoker will be deemed to have abstained from smoking. Suitably, the tobacco product or cigarette is a non-menthol tobacco product or non-menthol cigarette.

The term “purified” does not require absolute purity. Instead, it denotes a discrete environment in which abundance of the purified molecule (conveniently expressed in terms of mass or weight or concentration) relative to other molecules is greater than in a biological sample. A discrete environment denotes a single medium, such as for example a single solution, gel, precipitate, lyophilisate, etc. Purified molecules may be obtained by known methods including, for example, chromatography, preparative electrophoresis, centrifugation, precipitation, affinity purification, etc.

The terms “quantity”, “amount” and “level” are synonymous and generally well-understood in the art. With respect to metabolites, the terms may particularly refer to an absolute quantification of the metabolite in a sample, or to a relative quantification of the metabolite in the sample, i.e., relative to another value such as relative to a baseline or reference value as taught herein, or to a range of values indicating a base-line expression of the metabolite. These values or ranges may be obtained from a single subject or from a group of subjects. An absolute quantity of a metabolite in a sample may be advantageously expressed as weight or as molar amount, or more commonly as a concentration, for example, weight per volume or mol per volume.

The terms “sample” or “biological sample” as used herein include any biological specimen obtained from a subject. Samples may include, without limitation, whole blood, plasma, serum, red blood cells, white blood cells (for example, peripheral blood mononuclear cells), saliva, urine, stool (i.e., faeces), tears, sweat, sebum, nipple aspirate, ductal lavage, tumour exudates, synovial fluid, cerebrospinal fluid, lymph, fine needle aspirate, amniotic fluid, any other bodily fluid, nail clippings, cell lysates, cellular secretion products, inflammation fluid, vaginal secretions, or biopsies such as preferably placental biopsies. Preferred samples may include those comprising any one or more metabolites as taught herein in detectable quantities. In one embodiment, the sample may be whole blood or a fractional component thereof such as—plasma, serum, or a cell pellet. Preferably, the sample is readily obtainable by minimally invasive methods, allowing to detect, remove or isolate said sample from the subject. Samples may also include tissue samples and biopsies, tissue homogenates and the like. The term “plasma” generally denotes the substantially colourless watery fluid of the blood that contains no cells, but in which the blood cells (erythrocytes, leukocytes, thrombocytes, etc.) are normally suspended, containing nutrients, sugars, proteins, minerals, enzymes, etc. In a most suitable embodiment, the sample is urine—such as 24-hour urine as it is easy and non-invasive to obtain. A 24-hour urine collection is done by collecting a subject's urine in a container over a full 24-hour period.

The term “smoker” is indicative of a subject that has smoked 10 or more tobacco products (for example, cigarettes) per day on average in the past year. The smoker will generally be a current smoker. Suitably, the tobacco product or cigarette is a non-menthol tobacco product or non-menthol cigarette.

The term “switcher” is indicative of a subject that has switched from smoking combustible tobacco products (for example, cigarettes) to heat-not-burn (smoke-free) products—such as iQOS—during the past 3 months.

The term “subject” as used herein typically denotes humans, but may also encompass reference to non-human animals, preferably warm-blooded animals, more preferably viviparous animals, even more preferably mammals, such as—non-human primates, rodents, canines, felines, equines, ovines, porcines, and the like.

The term “threshold” in the context of detection means the point at which a certain or defined amount or quantity or concentration is reached or crossed. For example, a test line on an immunoassay device can be configured to produce a visual change when a threshold amount or quantity or concentration is reached or crossed. The visual change may be the appearance or disappearance of a test line.

### DETAILED DESCRIPTION

The antibodies or antigen binding fragments thereof described herein are capable of binding to N-acetyl-S-[2-carboxyethyl]-L-cysteine (CEMA). The antibodies or antigen binding fragments thereof described herein are capable of binding to a conjugate comprising the compound of formula [I] or [II] or [III] wherein the compound is coupled to an immunogenic carrier via a linker.

The antibodies or antigen binding fragments thereof described herein are capable of specifically binding to N-acetyl-S-[2-carboxyethyl]-L-cysteine (CEMA). The antibodies or antigen binding fragments thereof described herein are capable of specifically binding to the conjugate comprising the compound of formula [I] or [II] or [III], wherein the compound is coupled to an immunogenic carrier via a linker.

The antibody or antigen binding fragment therefore may bind with high affinity, for example, with a dissociation constant (K_{d}) of less than 1 μM, preferably less than 1 nM. Suitably, the antibody specifically binds to CEMA and the CEMA conjugate and does not significantly bind to Cotinine or molecules with a structure close to CEMA—such as 2-hydroxyethyl mercapturic acid (HEMA), monohydroxybutenylmercapturic acid (MHBMA), 3-hydroxypropyl mercapturic acid (3-HPMA) or dihydroxybutylmercapturic acid (DHBMA).

The respective quantities or measurements for the metabolites used as tobacco smoke exposure biomarkers as described herein may be evaluated simultaneously, separately or individually. Suitably, the metabolites described herein are evaluated substantially simultaneously or simultaneously. Suitably, the metabolites described herein are evaluated simultaneously at the same point in time. The amounts of the metabolite(s) can be used to establish if a subject is a current smoker of conventional cigarettes. The amounts of the metabolite(s) can be used to establish if a subject has abstained from smoking. The amounts of the metabolite(s) can be used to establish if a subject is a switcher from being a current smoker of conventional cigarettes to a consumer of a RRP. A switching profile can be established for the subject and switching compliance behaviour can be assessed over time to monitor the progression of the switching behaviour. Conveniently, the analysis can be carried out in a single test.

The metabolites can comprise or consist or consist essentially of CEMA or CEMA and Cotinine. In certain embodiments, it is intended to encompass isomeric forms (such as stereoisomers and/or geometric and/or optical isomers, and mixtures thereof), chemical derivatives, mimetics, variants, solvates and salts of these metabolites.

In certain embodiments, one or more additional metabolites—such as one or more other tobacco smoke exposure biomarkers—can also be tested simultaneously, separately and/or individually, if required. In addition, more general characteristics of a sample can be evaluated simultaneously, separately and/or individually. For example, to test for the adulteration or dilution of a sample, certain characteristics can be detected. In the case of a urine sample, characteristics—such as one or more of pH, specific gravity, oxidants, nitrite, glutaraldehyde and creatinine levels—may be measured in the urine sample.

CEMA is a specific urinary biomarker of acrolein exposure. Urinary excretion of CEMA has been shown to be consistently higher in smokers than in non-smokers. Ranges in non-smokers are usually below 2 ng/mL and increase to levels of more than 20 to 205 ng/mL in smokers. In one example, smokers excreted 187±181 μg/L (mean±SD) or 184 μg/L of CEMA, while non-smokers only excreted 4.6±35 μg/L or 1.9 μg/L. Smokers consuming 20 cigarettes per day (CPD) or more who were required to smoke exactly 20, 15, 5 or 0 CPD during a 5-day confinement period show a dose-relationship between the number of cigarettes and the levels of urinary CEMA (218.0, 168.0, 93.2 and 38.3 μg/24 h, respectively). Smoking cessation studies of 5 to 8 days show significantly lower levels of CEMA in urine by about 7- to 10-fold. CEMA can be detected using the antibody or antigen-binding fragment thereof of the present invention—such as via immunoassay. Various kinds of immunoassay technology are known in the art as described herein. Various methods are known in the art for measuring CEMA—such as ultra-high-performance liquid chromatography coupled with electrospray ionization tandem mass spectrometry. The determination of CEMA in urine has also been described in Anal. Bioanal. Chem. (2009) 393:969-981 and Anal Biochem. (2012) 430(1):75-82.

Another metabolite is Cotinine and is the main nicotine metabolite detected in the urine of smokers. Cotinine levels in various biological fluids are widely used to estimate intake of nicotine in tobacco users. Cotinine has an in vivo half-life of about 20 hours and can be detected for several days after the use cigarettes of tobacco use. The level of Cotinine in blood, saliva, and urine is proportionate to the amount of exposure to tobacco smoke. Cotinine levels of <10 ng/mL are indicative of no active smoking. Values of 10 ng/mL to 100 ng/mL are indicative of light smoking or moderate exposure. Levels above 300 ng/mL are indicative of heavy smoker status consuming more than 20 CPD. In urine, values between 11 ng/mL and 30 ng/mL are indicative of light or moderate smoking, and levels in active smokers are about 500 ng/mL or more. In saliva, values between 1 ng/mL and 30 ng/mL are indicative of light or moderate smoking and levels in active smokers are about 100 ng/mL or more. Cotinine can be detected using anti-Cotinine antibody or an antigen-binding fragment thereof—such as via immunoassay. Other methods for measuring Cotinine include colorimetric methods, gas chromatography (GC) and gas chromatography-mass spectrometry (GC-MS), high-performance liquid chromatography, and radioimmunoassay (RIA). Methods for measuring Cotinine are also described in BMB Rep. (2014) March; 47(3): 130-134; J Clin Diagn Res. (2016) March; 10(3): ZE04-ZE06 and Ther Drug Monit. (2009) February; 31(1): 14-30.

Antibodies are naturally occurring immunoglobulin molecules which have varying structures, all based upon the immunoglobulin fold. For example, IgG antibodies have two ‘heavy’ chains and two ‘light’ chains that are disulphide-bonded to form a functional antibody. Each heavy and light chain itself comprises a ‘constant’ (C) and a ‘variable’ (V) region. The V regions determine the antigen binding specificity of the antibody, whilst the C regions provide structural support and function in non-antigen-specific interactions with immune effectors. The antigen binding specificity of an antibody or antigen-binding fragment of an antibody is the ability of an antibody or antigen-binding fragment thereof to specifically bind to a particular antigen. The antigen binding specificity of an antibody is determined by the structural characteristics of the V region. The variability is not evenly distributed across the 110-amino acid span of the variable domains. Instead, the V regions consist of relatively invariant stretches called framework regions (FRs) of 15-30 amino acids separated by shorter regions of extreme variability called ‘hypervariable regions’ that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Each V region typically comprises three CDRs, each of which contains a ‘hypervariable loop’, and four framework regions. An antibody binding site, the minimal structural unit required to bind with substantial affinity to a particular desired antigen, will therefore typically include the three CDRs, and at least three, preferably four, framework regions interspersed there between to hold and present the CDRs in the appropriate conformation. Classical four chain antibodies have antigen binding sites which are defined by VH and VL domains in cooperation. Certain antibodies, such as camel and shark antibodies, lack light chains and rely on binding sites formed by heavy chains only. Single domain engineered immunoglobulins can be prepared in which the binding sites are formed by heavy chains or light chains alone, in absence of cooperation between VH and VL.

The antibody or antigen-binding fragment thereof described herein may be isolated or purified to any degree. In some embodiments, contaminant components of its natural environment are materials which would interfere with (diagnostic) uses for the antibody and may include enzymes and other proteinaceous or non-proteinaceous solutes. In some embodiments, the antibody or antigen-binding fragment will be purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or nonreducing conditions using Coomassie blue or, preferably, silver stain. Ordinarily, isolated antibody will be prepared by at least one purification step.

The antibody may be any of IgA, IgD, IgE, IgG and IgM classes, and preferably an IgG class antibody. The antibody may be a polyclonal antibody, for example, an antiserum or immunoglobulin purified there from (for example, affinity-purified). Suitably, the antibody is a monoclonal antibody or a mixture of monoclonal antibodies. Monoclonal antibodies can target a particular antigen or a particular epitope within an antigen with greater selectivity and reproducibility. By means of example and not limitation, monoclonal antibodies may be made by the hybridoma method first described by Kohler et al. (1975) Nature 256: 495, or using the trioma technique, or using the human B-cell hybridoma technique (Kozbor (1983) Immunology Today 4:72) and the EBV-hybridoma technique to produce human monoclonal antibodies (Cole et al., (1985) Monoclonal Antibodies and Cancer Therapy, Alan R. Liss, Inc. 77-96). DNA encoding the antibody or antigen-binding fragment can be sequenced providing for the information to recombinantly produce the antibody or antigen-binding fragment thereof in small or large scale. There is also disclosed a method for producing the antibodies comprising immunizing a non-human animal with a CEMA conjugate as described herein. Methods of producing antibodies and antigen-binding fragments thereof are well known in the art, as are methods to produce recombinant antibodies or antigen-binding fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921). Monoclonal antibodies may also be isolated from phage antibody libraries using techniques as described by, for example, Clackson et al. (1991) Nature 352: 624-628) and Marks et al. (1991) J. Mol. Biol. 222: 581-597.

Antibodies may be antigen-binding fragments thereof. Such fragments comprise a portion of an intact antibody, comprising the antigen-binding or variable region thereof. Examples of antigen-binding fragment include Fab, Fab′, F(ab′)2, Fv and scFv fragments; diabodies; linear antibodies; single-chain antibody molecules; and multivalent and/or multispecific antibodies formed from antigen-binding fragment(s), for example, diabodies, tribodies, and multibodies. A skilled person will understand that an antibody may include one or more amino acid deletions, additions and/or substitutions (for example, conservative substitutions), insofar as such alterations preserve its binding of CEMA or the CEMA conjugate. To preserve binding, such changes can be made to those parts of the amino acid sequence of the antibody that are not responsible for binding CEMA or the CEMA conjugate. An antibody may also include one or more native or artificial modifications of its constituent amino acid residues (for example, glycosylation), insofar as such alterations preserve its binding of CEMA or the CEMA conjugate.

The antibody or antigen-binding fragment thereof may be associated with or attached to a detection agent—such as a label—to facilitate detection. Examples of such detection agents include, but are not limited to, luminescent labels, colorimetric labels—such as dyes, fluorescent labels, or chemical labels—such as electroactive agents, enzymes, radioactive labels, or radiofrequency labels. Examples of detection agents include any composition detectable by spectroscopic, photochemical, biochemical, immunochemical, electrical, optical or chemical means. The detection agent can be a particle—such as a colloidal gold particle, colloidal sulphur particle, colloidal selenium particle, colloidal barium sulphate particle, colloidal iron sulphate particle, metal iodate particle, silver halide particle, silica particle, colloidal metal (hydrous) oxide particle, colloidal metal sulphide particle, colloidal lead selenide particle, colloidal cadmium selenide particle, colloidal metal phosphate particle, colloidal metal ferrite particle, any of the above-mentioned colloidal particles coated with organic or inorganic layers, protein or peptide molecules, liposomes, or organic polymer latex particles—such as polystyrene latex beads. More suitable and preferred particles are colloidal gold particles which are one of the most commonly used labels. Colloidal gold may be made by any conventional means—such as using the methods of G. Frens (1973) Nature Physical Science, 241:20 (1973). Alternative methods are described in U.S. Pat. Nos. 5,578,577, 5,141,850, 5,079,172, 5,202,267, 5,514,602, 5,616,467 and 5,681,775.

The detection agent may be a tag that permits detection with another agent—such as a binding partner. Such tags can be, for example, FLAG tag, biotin, streptavidin, his-tag, myc tag, maltose, maltose binding protein or any other kind of tag known in the art that has a binding partner. Examples of associations which can be utilised may include biotin:streptavidin, his-tag/metal ion or maltose/maltose binding protein. The use of a FLAG tag is preferred in certain embodiments.

In some embodiments, the antibody is labelled directly or indirectly to allow detection of metabolite in a sample. For example, the labelled antibody can be combined with the sample, and the labelled antibody-metabolite complex is detected.

In accordance with the present invention, CEMA can be detected using the antibody or an antigen-binding fragment thereof as described herein. In particular, the antibody or antigen binding fragment thereof is capable of binding CEMA and is capable of binding to a conjugate comprising a compound of formula [II]:

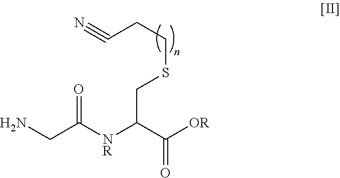

wherein n is selected from 0 to 4 (that is, 0, 1, 2, 3, or 4), and each R is independently selected from H or C₁ to C₆ alkyl; preferably, a compound of formula [I]:

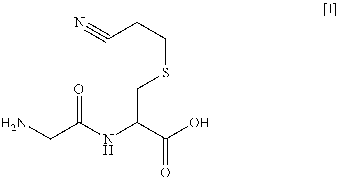 [SMILES: N#CCCSCC(NC(=O)CN)C(=O)O]

wherein the compound of formula [II] or formula [I] is coupled to an immunogenic carrier via a linker. In one embodiment, the linker is coupled to the compound of formula [I] via the amine group.

In one embodiment, the compound of formula [I] has the structure of the compound of formula [III]:

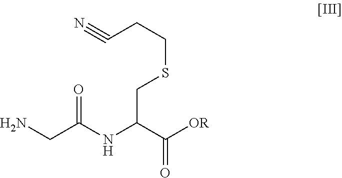

wherein R is selected from H and C₁ to C₆ alkyl.

Three such antibodies of the present invention have been extensively characterised and are denoted herein as ‘B11 scFv’. ‘E6 scFv’ and ‘G4 scFv’ (and may also be referred to herein as ‘B11’, ‘E6’ and ‘G4’). The polypeptide sequences of the three CDRs of each of the variable domains of the VL and VH chains for each of B11 scFv, E6 scFv and G4 scFv are described herein.

The VH CDR1, VH CDR2 and VH CDR3 of the B11 scFv correspond to the amino acid sequences of SEQ ID NO:3, SEQ ID NO:4 and SEQ ID NO:5, respectively. The VL CDR1, VL CDR2 and VL CDR3 of the B111 scFv correspond to the amino acid sequences of SEQ ID NO:7, SEQ ID NO:8 and SEQ ID NO:9, respectively.

The VH CDR1, VH CDR2 and VH CDR3 of the E6 scFv correspond to the amino acid sequences of SEQ ID NO:12, SEQ ID NO:13 and SEQ ID NO:14, respectively. The VL CDR1, VL CDR2 and VL CDR3 of the E6 scFv correspond to the amino acid sequences of SEQ ID NO:16, SEQ ID NO:17 and SEQ ID NO:18, respectively.

The VH CDR1, VH CDR2 and VH CDR3 of the G4 scFv correspond to the amino acid sequences of SEQ ID NO:21, SEQ ID NO:22 and SEQ ID NO:23, respectively. The VL CDR1, VL CDR2 and VL CDR3 of the G4 scFv correspond to the amino acid sequences of SEQ ID NO:25, SEQ ID NO:26 and SEQ ID NO:27, respectively.

The antibody or antigen binding fragment thereof can comprise a VH amino acid sequence consisting of the amino acid sequence of SEQ ID NO: 2 or 11 or 20.

The antibody or antigen binding fragment thereof can comprise a VL amino acid sequence consisting of the amino acid sequence of SEQ ID NO:6 or 15 or 24.

The antibody or antigen binding fragment thereof can comprise a VH amino acid sequence consisting of the amino acid sequence of SEQ ID NO: 2 and a VL amino acid sequence consisting of the amino acid sequence of SEQ ID NO:6. These are the VH and VL amino acid sequences of B11 scFv.

The antibody or antigen binding fragment thereof can comprise a VH amino acid sequence consisting of the amino acid sequence of SEQ ID NO:11 and a VL amino acid sequence consisting of the amino acid sequence of SEQ ID NO:15. These are the VH and VL amino acid sequences of E6 scFv.

The antibody or antigen binding fragment thereof can comprise a VH amino acid sequence consisting of the amino acid sequence of SEQ ID NO:20 and a VL amino acid sequence consisting of the amino acid sequence of SEQ ID NO:24. These are the VH and VL amino acid sequences of G4 scFv.

There is also disclosed a scFv comprising or consisting of the amino acid sequence of SEQ ID NO: 1 (B11 scFv) or SEQ ID NO: 10 (E6 scFv) or SEQ ID NO: 19 (G4 scFv).

A detailed analysis of the amino acid sequence of B11, E6 and G4 scFv according to Kabat numbering is presented in Table 1.

The polynucleotide sequences of each of the variable domains of the VL and VH chains for each of B11 scFv, E6 scFv and G4 scFv are also described herein.

The VH of the B11 scFv can be encoded by the polynucleotide sequence comprising or consisting of SEQ ID NO:29. The VL of the B11 scFv can be encoded by the polynucleotide sequence comprising or consisting of SEQ ID NO:30.

The VH of the E6 scFv can be encoded by the polynucleotide sequence comprising or consisting of SEQ ID NO:32. The VL of the B11 scFv can be encoded by the polynucleotide sequence comprising or consisting of SEQ ID NO:33.

The VH of the G4 scFv can be encoded by the polynucleotide sequence comprising or consisting of SEQ ID NO:35. The VL of the B11 scFv can be encoded by the polynucleotide sequence comprising or consisting of SEQ ID NO:36.

The antibody or antigen binding fragment thereof can comprise a VH encoded by the polynucleotide sequence comprising or consisting of SEQ ID NO: 29 or SEQ ID NO: 32 or SEQ ID NO: 35.

The antibody or antigen binding fragment thereof can comprise a VL encoded by the polynucleotide sequence comprising or consisting of SEQ ID NO: 30, SEQ ID NO: 33 or SEQ ID NO: 36.

There is also disclosed a scFv that can be encoded by the polynucleotide sequence comprising or consisting of SEQ ID NO: 28 (B11 scFv) or SEQ ID NO: 31 (E6 scFv) or SEQ ID NO: 34 (G4 scFv).

In certain embodiments, B11 and G4 are preferred. In certain embodiments, B11 is preferred.

As the skilled person will appreciate, there are various models for assigning/identifying the CDR sequences in antibody VL/VH chains. The most popular/widely accepted versions are the Chothia and Kabat models, although others also exist such as the ABM and CONTACT models. The CDR sequences presented herein were determined using the Kabat model (Kabat et al., (1991) Sequences of Proteins of Immunological Interest, 5th Ed. Public Health Service, National Institutes of Health, Bethesda, Md.) as is conventional in the art.

Any one of a variety of immunogenic carrier proteins may be used for the conjugate of the present invention, for example, a protein, a peptide, an oligonucleotide or a polymer. The immunogenic carrier protein is coupled to a linker. Specific examples are albumins, such as bovine serum albumine (BSA), globulins, thyroglobulin, hemoglobins, hemocyanins, polylysine, polyglutamic acid, lysine-glutamic acid copolymers and copolymers containing lysine or omithine. Classes of suitable proteins include pili, outer membrane proteins and excreted toxins of pathogenic bacteria, nontoxic or ‘toxoid’ forms of such excreted toxins, nontoxic proteins antigenically similar to bacterial toxins and other proteins. The use of viral proteins is also contemplated. In a preferred embodiment, the immunogenic carrier is a protein, suitably, BSA or bovine thyroglobulin (BTG).

In one aspect, there is disclosed a method for the preparation of the conjugate described above, comprising: (a) activating the immunogenic carrier; and (b) coupling the activated immunogenic carrier obtained in step (a) to the compound of formula (I) via a linker.

In the present invention, the compound of formula (I) is linked to the immunogenic carrier protein, which involves the use of the primary amine (—NH2) group of the compound of formula (I). There are numerous synthetic chemical groups that will form chemical bonds with primary amines. These include isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters. Most of these conjugate to amines by either acylation or alkylation.

In one embodiment, the linker disuccinimidyl suberate (DSS) is used which is a non-cleavable and membrane permeable crosslinker that contains an amine-reactive N-hydroxysuccinimide (NHS) ester at each end of an 8-carbon spacer arm. NHS esters react with primary amines at pH 7-9 to form stable amide bonds, along with release of the N-hydroxysuccinimide leaving group. DSS is first dissolved in an organic solvent such as DMF or DMSO, then added to the aqueous crosslinking reaction.

In another embodiment, the linker ethylene glycol bis(succinimidyl succinate) (EGS) is used. EGS is a water-insoluble, homobifunctional N-hydroxysuccinimide ester (NHS ester). The spacer arm contains two cleavable ester sites that may be broken with hydroxylamine, which yields two fragments with terminal amide bonds and the release of ethylene glycol. Accessible α-amine groups present on the N-termini of proteins and peptides and ε-amine of lysine react with NHS esters at pH 7-9 to form covalent amide bonds. The reaction results in the release of N-hydroxysuccinimide. NHS ester crosslinking reactions are most commonly performed in phosphate, carbonate/bicarbonate. HEPES and borate buffers.

For the preparation of the conjugate the skilled person will readily be able to determine suitable conjugation methods, for example, as found in Hermanson, G. T. (1996) Bioconjugate Techniques 1st ed.; Academic Press: San Diego, California, Vol. 1.

In one embodiment, the conjugate (immunogen) comprises a compound of formula [I]:

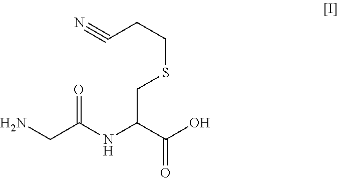 [SMILES: N#CCCSCC(NC(=O)CN)C(=O)O]

wherein the compound of formula [I] is coupled to BTG via an EGS linker that is coupled to the amine group of the compound of formula [I]. This compound is referred to herein as ‘BTG-EGS-CEMA’.

In another embodiment, the conjugate (immunogen) comprises a compound of formula [I]:

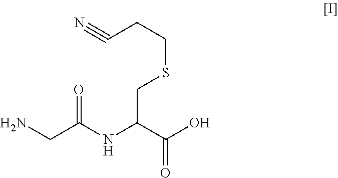 [SMILES: N#CCCSCC(NC(=O)CN)C(=O)O]

wherein the compound of formula [I] is coupled to an BTG via a DSS linker that is coupled to the amine group of the compound of formula [I]. This conjugate is referred to herein as ‘BTG-DSS-CEMA’.

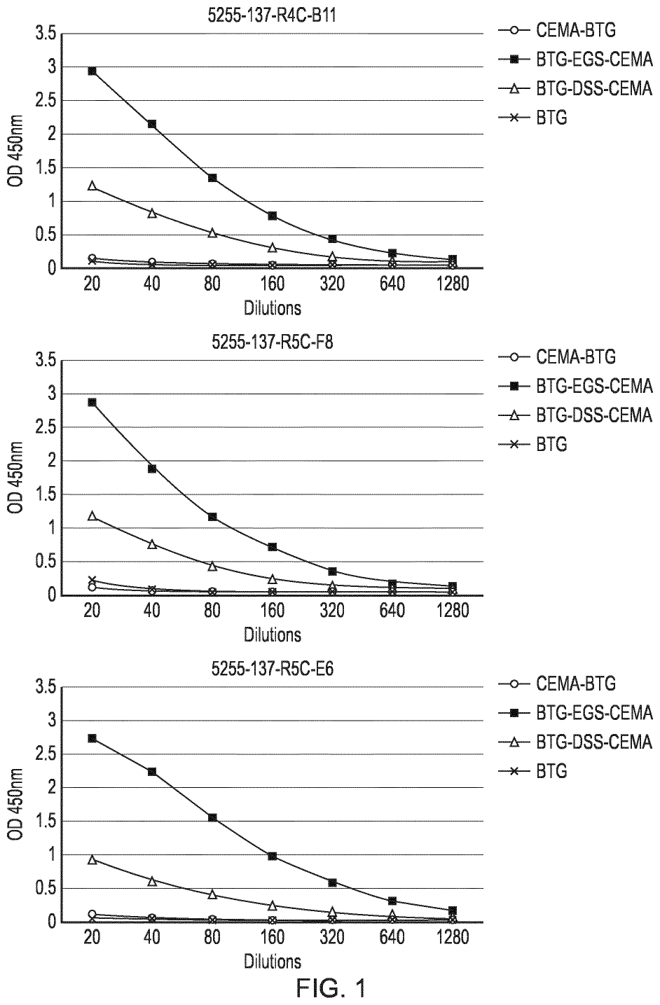

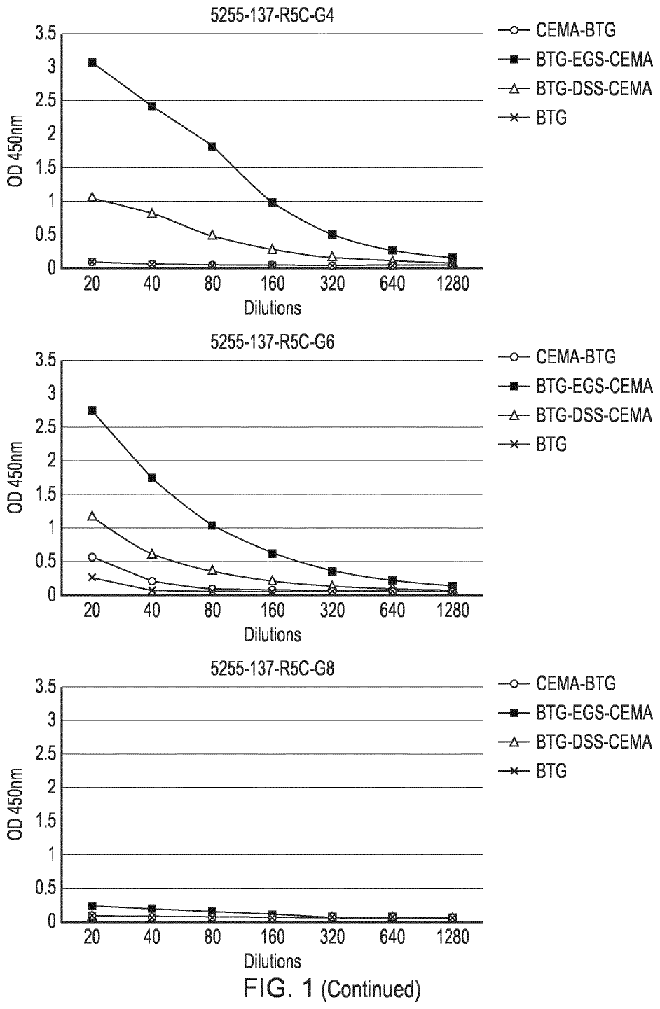

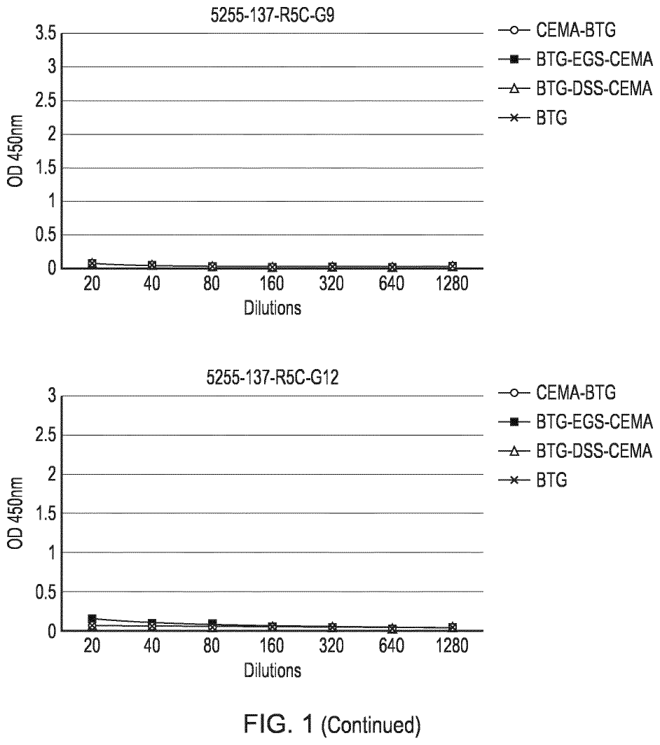

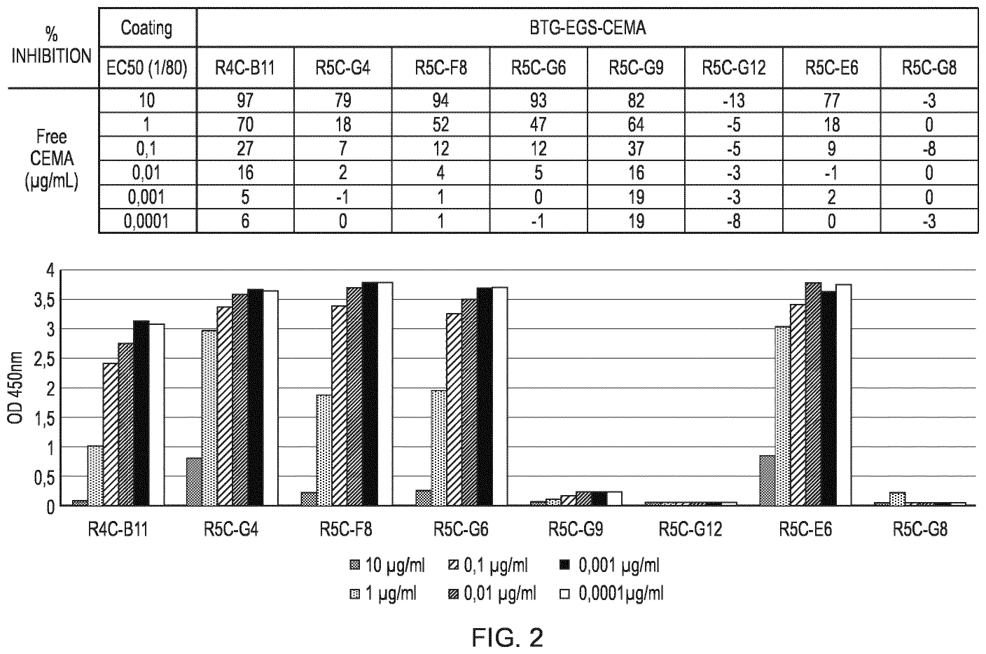

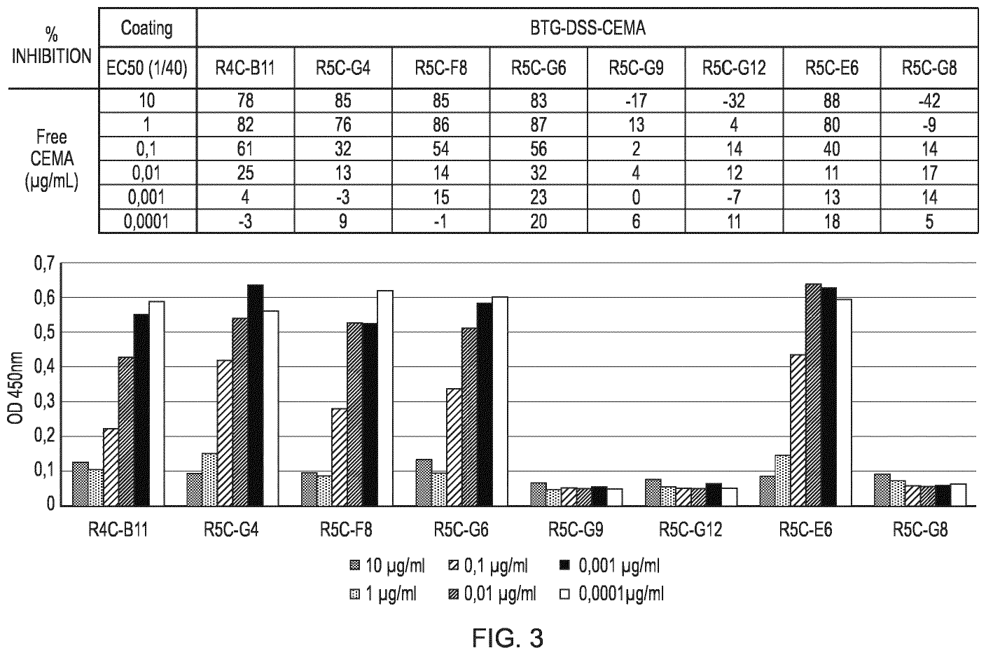

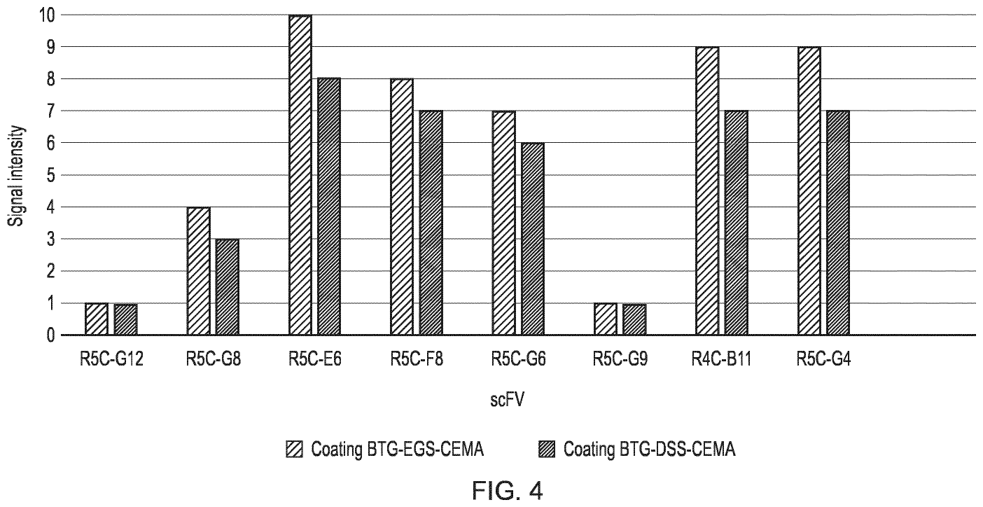

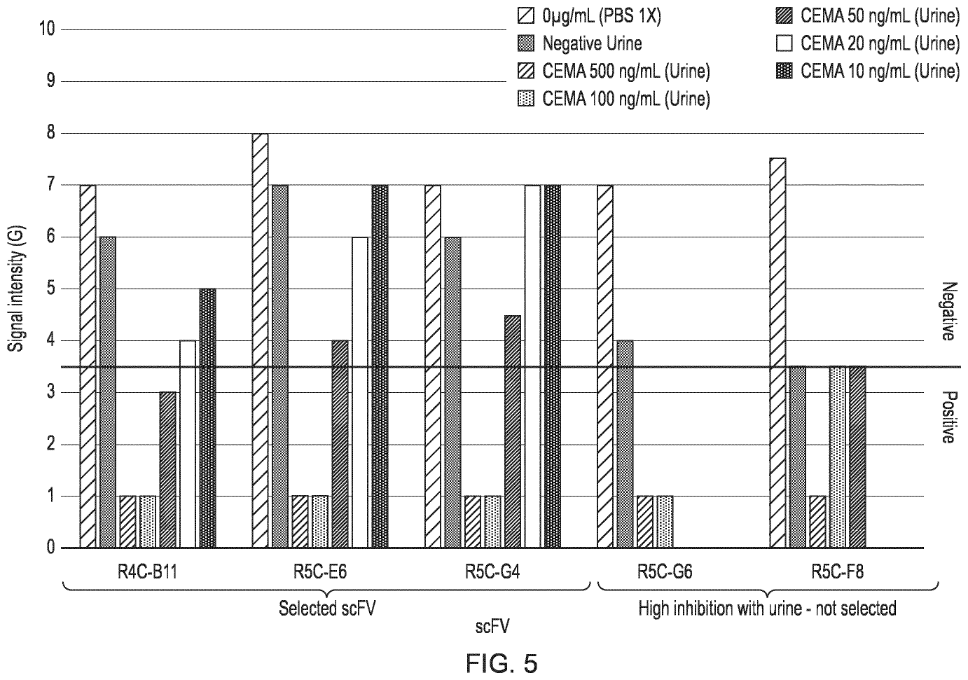

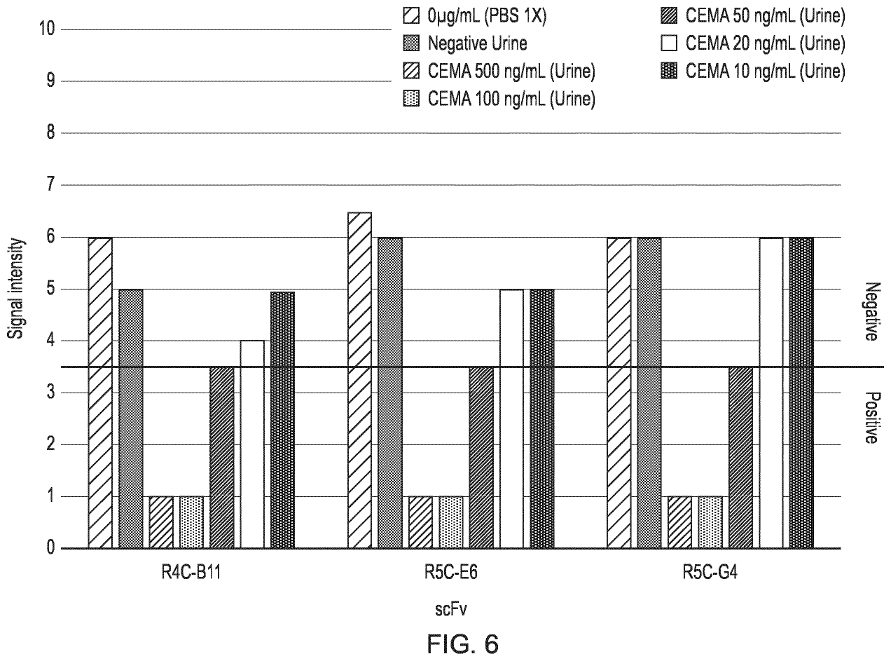

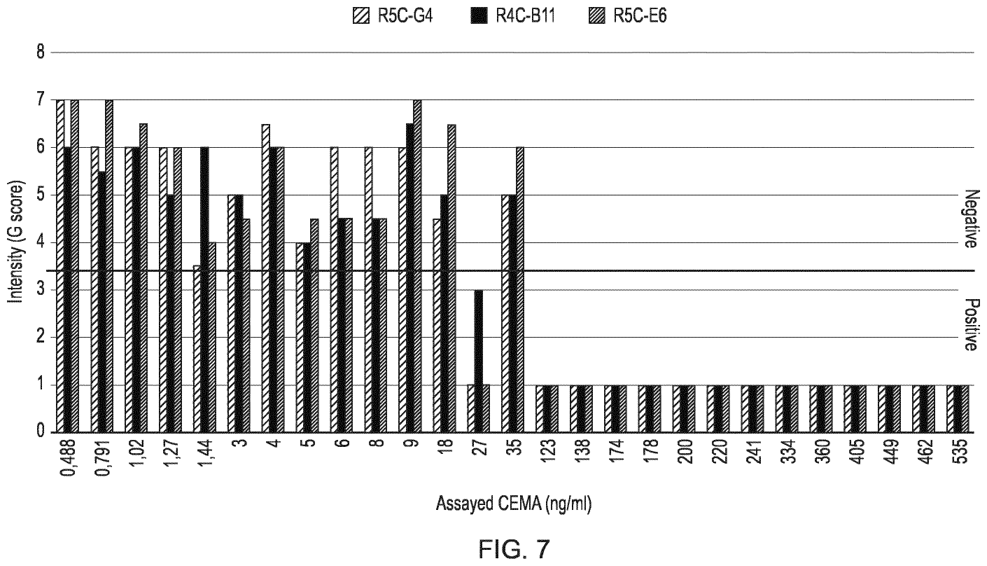

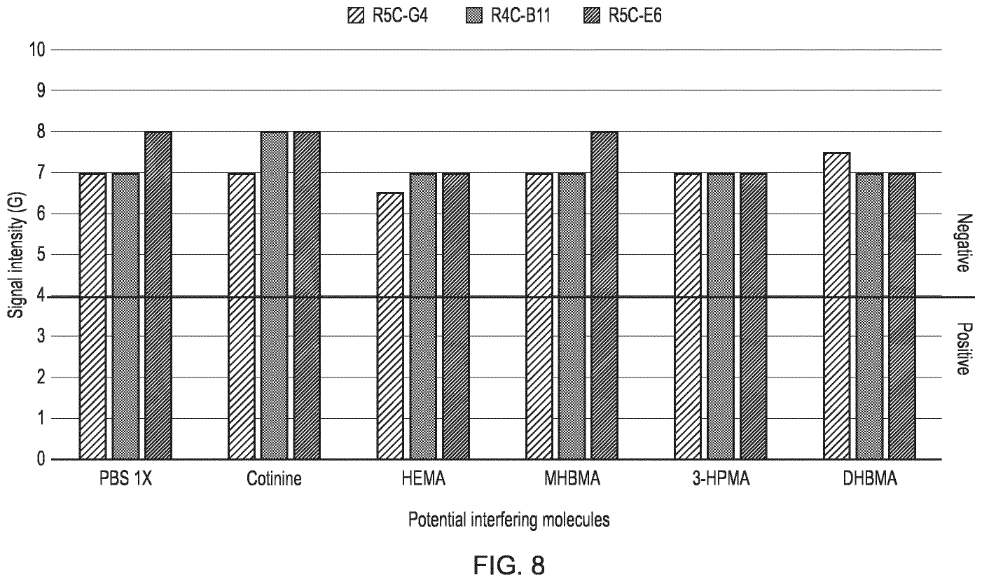

The compound of formula [I] ensures an intact CEMA structure after coupling to the protein. In an embodiment of the invention, it is preferred that the antibody of the present invention is an antigen-binding fragment. Suitably, the antigen-binding fragment is an scFv. According to the present disclosure, scFv were identified by screening ELISA-phage against the CEMA conjugate described herein and of 96 colonies that were picked, 28 ‘hits’ with high signal were identified as positives. DNA was extracted and sequenced and 8 non-redundant sequences were identified which were then used to produce 8 scFv which were tested in ELISA and lateral flow immunoassay for CEMA binding. 3 scFv were selected (denoted as G4 scFv, B11 scFv and E6 scFv herein) based on the following highly advantageous properties:
1) reactivity on CEMA-carrier haptens. As can be appreciated from FIG. 1, G4, B11 and E6 have good reactivity with BTG-EGS-CEMA and BTG-DSS-CEMA as compared to CEMA-BTG and BTG alone in ELISA assays.
2) binding inhibition in competitive ELISA using BTG-EGS-CEMA and free CEMA. As can be appreciated from FIG. 2, G4, B11 and E6 have acceptable % inhibition at 10 μg/mL free CEMA with 1 μg/ml free CEMA discriminating the sensitivity.
3) binding inhibition in competitive ELISA using BTG-DSS-CEMA and free CEMA. As can be appreciated from FIG. 3, G4, B11 and E6 have acceptable % inhibition at 10 μg/mL free CEMA and 1 μg/ml free CEMA with 0.1 ug/ml free CEMA discriminating the sensitivity.
4) reactivity testing of scFv via lateral flow immunoassay. As can be appreciated from FIG. 4, G4, B11 and E6 are recognised with the immunogens BTG-EGS-CEMA and BTG-DSS-CEMA. The scFv G12 and G9 are not recognized by either immunogen and scFv G8 had low recognition. Acceptable reactivity testing was obtained for the scFv E6, F8, G6, B11 and G4.
5) cross-reaction with urine. As can be appreciated from FIG. 5, good inhibition (indicative of positive signal) is achieved until 100 ng/mL urine with B11, E6 and G4 on the immunogen BTG-EGS-CEMA. The signal is slightly decreased in the presence of urine but not inhibited. As can be appreciated from FIG. 6, good inhibition (indicative of positive signal) is also achieved until 100 ng/mL urine with B11, E6 and G4 on the BTG-DSS-CEMA immunogen. The signal is slightly decreased in the presence of urine but not inhibited. As can be appreciated, B11, E6 and G4 allow the detection of all positive urine with CEMA above 100 ng/mL.
6) As can be appreciated from FIG. 7, B11, E6 and G4 allow the detection of all positive urine with CEMA above 100 ng/mL.
7) As can be appreciated from FIG. 8, no cross reactivity is seen with Cotinine or compounds with a structure close to CEMA, including 2-hydroxyethyl mercapturic acid (HEMA), monohydroxybutenylmercapturic acid (MHBMA), 3-hydroxypropyl mercapturic acid (3-HPMA) or dihydroxybutylmercapturic acid (DHBMA) using a tested concentration of 1 μg/ml. G4, B11 are highly specific for CEMA.

Based on this experimental data, it was concluded that G4, B11 and E6 are excellent scFv for detecting CEMA, especially in urine. Thus, in a preferred embodiment, the antibody is an antigen-binding fragment thereof. In a more preferred embodiment, the antigen-binding fragment is an scFv fragment. In a more preferred embodiment, the antigen-binding fragment is scFv G4, scFv B11 and scFv E6 as defined herein.

F(ab′)2 (110,000 daltons) fragments contain two antigen-binding regions joined at the hinge through disulfides. This fragment is void of most, but not all, of the Fc region.

Fab′ (55,000 daltons) fragments can be formed by the reduction of F(ab′)2 fragments. The Fab′ fragment contains a free sulfhydryl group that may be alkylated or utilised in conjugation with an enzyme, toxin or other protein of interest. Fab′ is derived from F(ab′)2; therefore, it may contain a small portion of Fc.

Fab (50,000 daltons) is a monovalent fragment that can be produced from IgG and/or IgM, consisting of the VH, CH1 and VL. CL regions, linked by an intramolecular disulfide bond.

Fv (25,000 daltons) is the smallest fragment produced from IgG and/or IgM that contains a complete antigen-binding site. Fv fragments have the same binding properties and similar three-dimensional binding characteristics as Fab. The VH and VL chains of the Fv fragments are held together by non-covalent interactions. These chains tend to dissociate upon dilution, so methods have been developed to cross-link the chains through glutaraldehyde, intermolecular disulfides or a peptide linker.

scFv are single chain Fvs and can be conveniently made recombinantly. The molecular weight is approximately 28,000 Da. It consists of the variable regions of the VH and VL of immunoglobulins, connected with a short linker peptide of 10-25 amino acids. Each VH and VL domain contains three CDRs. To make scFv, mRNA is first isolated from hybridoma (or spleen, lymph cells, and bone morrow) followed by reverse transcription into cDNA to serve as a template for antibody gene amplification using, for example, PCR. With this methodology, large libraries with a diverse range of antibody VH and VL genes can be created. Biopanning, which is the procedure of selecting binding partners from phage display libraries, is used to obtain the scFv with the best affinity and specificity. Once antibody genes are successfully cloned and sequenced, scFv fragments can be readily expressed in suitable expression systems. A suitable purification tag is typically added to the C-terminus of the antibody scFv fragment—such as ploy-histidine tag, FLAG-tag, HA-tag, and Myc-tag. A protease cleavage size can be designed to allow tag-removal after purification. Alternatively, the tag can be retained and utilised in immunoassay. Further details on scFv can be found in Ahmad et al. (2012) Clinical and developmental immunology (2012), Biosensors and Bioelectronics (2016) 85, 32-45. MAbs (2010) 2(1) 77-83 and U.S. Pat. No. 4,946,778.

Also provided herein are isolated nucleic acids encoding the antibodies and antigen-binding fragments thereof, vectors and host cells comprising the nucleic acid, and recombinant techniques for the production of the antibody or antigen-binding fragments thereof. The antibodies described herein can be produced by recombinant expression. Nucleic acids encoding the light and heavy chain variable regions are optionally linked to constant regions, and inserted into an expression vector(s). The light and heavy chains can be cloned in the same or different expression vectors. The DNA segments encoding immunoglobulin chains are operably linked to control sequences in the expression vector(s) that ensure the expression of immunoglobulin polypeptides. Expression control sequences include, but are not limited to, promoters, signal sequences, enhancer elements, and transcription termination sequences. Once the expression vector has been incorporated into the appropriate host, the host is maintained under conditions suitable for high level expression of the nucleotide sequences, and the collection and purification of the cross-reacting antibodies or antigen-binding fragments thereof. Commonly, expression vectors contain selection markers to permit detection of those cells transformed with the desired DNA sequences. The antibody or antigen-binding fragments thereof may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (that is, cleaved by a signal peptidase) by the host cell. When heavy and light chains are cloned on separate expression vectors, the vectors are co-transfected to obtain expression and assembly of intact immunoglobulins. Once expressed, the whole antibodies, their dimers, individual light and heavy chains, or other immunoglobulin forms can be purified. Substantially pure immunoglobulins of at least about 90 to 95% homogeneity are preferred, and 98 to 99% or more homogeneity is most preferred.

When using recombinant techniques, the antibody or antigen-binding fragment thereof can be produced intracellularly, in the periplasmic space, or directly secreted into the medium. If the antibody or antigen-binding fragment thereof is produced intracellularly, as a first step, the particulate debris, either host cells or lysed fragments, are removed, for example, by centrifugation or ultrafiltration. Carter et al., (1992) Bio/Technology 10: 163-167 describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 minutes. Cell debris can be removed by centrifugation. Where the antibody or antigen-binding fragment thereof is secreted into the medium, supernatants from such expression systems are generally first concentrated using a commercially available protein concentration filter, for example, an Amicon or Millipore Pellicon ultrafiltration unit. A protease inhibitor—such as PMSF—may be included in any of the foregoing steps to inhibit proteolysis and antibiotics may be included to prevent the growth of adventitious contaminants. The antibody or antigen-binding fragment thereof prepared from the cells can be purified using, for example, hydroxylapatite chromatography, gel electrophoresis, dialysis, and affinity chromatography, with affinity chromatography being the preferred purification technique. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody or antigen-binding fragment thereof to be recovered.

Immunoassay can be used to detect one or more of the metabolites described herein—including CEMA—using the antibody or antigen-binding fragment thereof of the present invention. Immunoassay technologies are known in the art and include direct ELISA (enzyme-linked immunosorbent assay), indirect ELISA, sandwich ELISA, competitive ELISA, multiplex ELISA, radioimmunoassay (RIA) technologies, fluorescent immunoassay, chemiluminescent immunoassay, DRI immunoassay, quantitative immunoassay, lateral flow immunoassay, microfluidic immunoassay and agglutination immunoassay and other similar techniques known in the art. Principles of these immunoassay methods are described in, for example, John R. Crowther, “The ELISA Guidebook”, 1st ed., Humana Press 2000, ISBN 0896037282. Additional information about practical immunochromatography can be found in the handbook “Lateral flow immunochromatography assays” by P. J. Davies et al. published on 15 Mar. 2008 by Wiley Online Library.

Direct ELISA employs a labelled primary antibody or an antigen-binding fragment thereof to bind to and thereby quantify target antigen in a sample immobilised on a solid support such as a microwell plate.

Indirect ELISA uses a non-labelled primary antibody or antigen-binding fragment thereof which binds to the target antigen and a secondary labelled antibody or antigen-binding fragment thereof that recognises and allows to quantify the antigen-bound primary antibody or antigen-binding fragment thereof.

In sandwich ELISA, the target antigen is captured from a sample using an immobilised ‘capture’ antibody which binds to one antigenic site within the antigen and, subsequent to removal of non-bound metabolite(s), the so-captured antigen is detected using a ‘detection’ antibody which binds to another antigenic site within said antigen, where the detection antibody may be directly labelled or indirectly detectable as above.

Competitive ELISA, which is a preferred immunoassay, uses a labelled ‘competitor’ that may either be the primary antibody or the target antigen. In an example, non-labelled immobilised primary antibody is incubated with a sample, this reaction is allowed to reach equilibrium and then labelled target antigen is added. The latter will bind to the primary antibody wherever its binding sites are not yet occupied by non-labelled target antigen from the sample. Thus, the detected amount of bound labelled antigen inversely correlates with the amount of non-labelled antigen in the sample.

Multiplex ELISA allows simultaneous detection of two or more metabolites within a single compartment usually at a plurality of array addresses (see, for example, Nielsen & Geierstanger 2004. J Immunol Methods 290: 107-20 and Ling et al. 2007. Expert Rev Mol Diagn 7: 87-98 for further guidance).

As will be appreciated, labelling in ELISA technologies is usually by enzyme conjugation and the end-point is typically colourimetric, chemiluminescent or fluorescent, magnetic, piezo electric, pyroelectric and other.

Devices for immunoassays are commonly suitable for single and domestic use because they are easy and quick to use and the results can be visualised using the naked eye. In some circumstances, more complex devices may be required for determining the presence and/or quantity of the metabolite(s), for example, when interpretation with the naked eye is either not possible or creates uncertainty. Such a device is described in WO2016/075405.

Radioimmunoassay (RIA) can be used to detect one or more of the metabolites described herein—including CEMA. This is a competition-based technique and involves mixing known quantities of radioactively-labelled (for example, ¹²⁵I- or ¹³¹I-labelled) target antigen with antibody or a fragment thereof to said antigen, then adding non-labelled or ‘cold’ antigen from a sample and measuring the amount of labelled antigen displaced (see “An Introduction to Radioimmunoassay and Related Techniques”, by Chard T, ed., Elsevier Science 1995, ISBN 0444821198).

Agglutination immunoassays can be used to detect one or more of the metabolites described herein—including CEMA. These assays utilise the binding and agglutination (clumping) of antibodies or fragments thereof to antigen-DNA conjugates, enabling ligation of the DNA strands and subsequent quantification by methods including quantitative polymerase chain reaction (qPCR).

Suitably, immunochromatography is used to detect one or more of the metabolites described herein—including CEMA. This is also known as a lateral flow immunochromatographic assay. Immunochromatography can be integrated into simple devices intended to detect the presence (or absence) of a metabolite in a sample—such as urine—without the need for specialised and costly equipment. Immunochromatography can also be integrated into portable lateral flow immunoassay devices, as discussed below.

The general principle of immunochromatography is based on a liquid sample containing or suspected of containing one or more of the metabolites to be detected via capillary action without the assistance of external forces through various zones of a lateral flow test strip. The lateral flow test strip generally comprises (i) a sample pad; (ii) a conjugate pad; (iii) a detection zone; and (iv) an optional absorption pad. The sample pad and optional absorption pad are located at opposing ends of the lateral flow test strip. Typically, the conjugate pad is adjacent the sample pad, the detection zone is adjacent the conjugate pad and the optional absorption pad is adjacent the detection zone.

The first element of the lateral flow test strip is a porous element for the sample which is referred to herein as the sample pad. This acts as a sponge and holds an excess of sample fluid. It is typically made of cellulose or glass fiber or a combination thereof. Its function is to transport the sample to other components of the lateral flow test strip. The sample pad should be capable of transportation of the sample in a smooth, continuous and homogenous manner. The sample pad can be impregnated with solutions—such as buffer salts and surfactants—as required. Once soaked, the fluid migrates to the second element of the lateral flow test strip. The second element of the lateral flow test strip is a porous element for conjugate and is referred to herein as a conjugate pad. This is where labelled antibody or antigen-binding fragments thereof, including those that are capable of individually binding CEMA (or in certain assay formats, CEMA analogues) and, optionally other metabolites, are present. Glass fiber, cellulose and polyesters are typical examples of materials used to make the conjugate pad. The labelled antibody or antigen-binding fragment thereof can be present in a dried format in a matrix, for example, a salt-sugar matrix, in the conjugate pad. In certain embodiments, two, three of four or more different antibodies or antigen-binding fragments thereof can be used when it is desired to detect metabolites in addition to CEMA. The amounts thereof can be adjusted as required to fine-tune the sensitivity of the assay for each metabolite. Typically, the amount of labelled antibody or antigen-binding fragments thereof that are used will be different for different metabolites. The labelled antibody or antigen-binding fragments thereof can be labelled with the same or a different label, as required. The sample fluid solubilises the labelled antibody or antigen-binding fragments thereof and in one combined transport action the sample and labelled antibody or antigen-binding fragments thereof mix while flowing through the conjugate pad to form the labelled antibody of antigen-binding fragments thereof-metabolite conjugate. The lateral flow test strip will have one or more detection zones—such as test lines—where another molecule(s) is immobilised. Typically, the detection zone will be a nitrocellulose membrane. The exact configuration of the detection zone will depend upon the format of the assay. The determination of a positive or negative result can be made using the naked eye or with a reader if a numeric or automated result is required. For example, the reader can be a camera—such as a charged coupled device camera or an optical sensor—such as a confocal optical sensor.

The competitive format is the generally preferred format according to the present invention since it is typically used for smaller metabolites—such as CEMA—that have fewer binding sites. The sample first encounters labelled or tagged antibodies to the target metabolite. In embodiments where tagged antibodies are used then detection will be permitted by the tagged antibodies which will be labelled. The test line contains the target metabolite fixed to the surface. When the target metabolite is absent from the sample, unbound antibody will bind to these fixed metabolite molecules, meaning that a visual marker will show. Conversely, when the target metabolite is present in the sample, it binds to the antibodies to prevent them binding to the fixed metabolite in the test line, and thus no visual marker shows. In one embodiment of the device configured for use in the competitive format: (a) the conjugate pad comprises or consists of labelled antibodies to the target metabolite(s) deposited thereon; and (b) the detection zone comprises or consists of the target metabolite(s) fixed to the surface, which can include CEMA or the CEMA conjugate as described herein. In another embodiment of the device configured for use in the competitive format utilising a tag: (a) the conjugate pad comprises or consists of a labelled anti-tag antibody deposited thereon; (b) an intermediate pad contains tagged antibodies or antigen binding fragments thereof to the target metabolite(s) deposited thereon, said tagged antibodies including tagged CEMA antibodies or antigen binding fragments as described herein; and (b) the detection zone comprises or consists of the target metabolite(s) fixed to the surface, which can include CEMA or the CEMA conjugate as described herein.

Suitably, the intermediate pad contains only tagged antibody or antigen binding fragments thereof to CEMA therein. The separate location of the intermediate pad allows fine adjustment of the amount of CEMA tagged antibody or antigen binding fragment thereof separate to the conjugate pad when more than one metabolite is being detected.

After passing through the conjugate pad and the detection zones, the fluid can enter the final porous element, which is referred to herein as the absorption pad. This functions as a waste container and is an optional feature. The various parts of the lateral flow test strip are fixed or mounted over a backing card which serves as a support and makes it easier to handle the strip.

A multiplex format can also be used when detecting more than one metabolite with a device. The multiplex detection format can be built in various ways, for example, by increasing the length of the lateral flow test strip or the length of the detection zone. The multiplex format can be used with the competitive format. In certain embodiments, a multiplex competitive format is preferred.

To obtain a qualitative or semi-quantitative result in which a signal is formed once the level of the metabolite(s) in the sample is higher than a certain predetermined threshold level or reference value or baseline value. Depending on the assay format, the intensity of the colour or signal may can be compared to a reference colour or signal chart. Alternatively, the amount or intensity of the colour or signal may be measured with an electronic device comprising, for example, a light absorbance sensor or light emission meter, resulting in a numerical value of signal intensity or colour absorbance formed. This embodiment can be of relevance for monitoring the level of said metabolite(s) in a subject over a period of time. It is also possible to measure the intensity of the detection zone to determine the quantity of metabolite in the sample, if required. Handheld diagnostic devices known as lateral flow readers are used by several companies to provide a fully quantitative assay result. One such handheld lateral flow device platform is made by Detekt Biomedical LLC.

The lateral flow test strip can be configured to include a positive control to demonstrate that the test works for subjects who do not have any metabolite in their urine (using, for example, creatine, albumin or a urine specific protein—such as Tamm-Horsfall protein (THP) as a marker(s)). In one embodiment the use of lateral flow immunoassay is preferred. In another embodiment the use of competitive format lateral flow immunoassay is preferred.

In another embodiment, the disclosure provides a portable lateral flow immunoassay device—such as a dipstick—to detect one or more of the metabolites described herein. The device uses the principle of immunochromatography as discussed above.

The portable lateral flow immunoassay device will typically comprise the lateral flow test strip contained in a housing—such as a liquid-tight or impervious housing—to allow the immersion of the device into a sample and to allow for only those necessary elements of the lateral flow test strip to be wetted. The device will normally have an elongated shape, the dimensions of which can vary, depending on the actual use of the device; exemplary dimensions are from 6 to 8 cm in length, and 3 to 6 mm in width.

Examples of portable lateral flow immunoassay devices are described in WO2007/023372, EP1657550, US2015/168397.

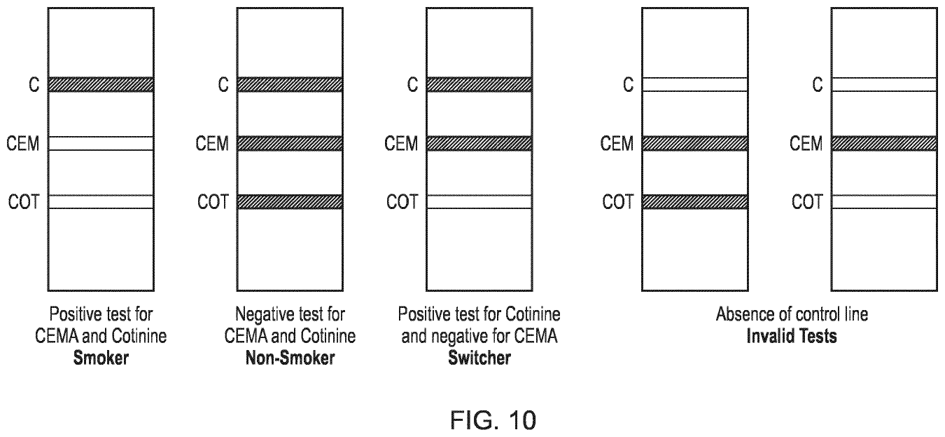

FIG. 10 depicts a preferred portable lateral flow immunoassay device or dipstick 100 configured in a competitive assay format. The device or dipstick 100 is configured to detect CEMA and Cotinine in this embodiment. In other embodiments, the device or dipstick 100 can be configured to detect CEMA and metabolites other than cotinine. In other embodiments, the device or dipstick 100 can be configured to detect only CEMA. The device 100 is made from a PVC support 109 to confer rigidity and has a nitrocellulose membrane 110 mounted thereon. Starting at the proximal end, a sample pad 102 for deposition of the sample to be tested is present and moving towards the distal end of the device, there is shown a conjugate pad 103 adjacent the sample pad, an intermediate pad 104 adjacent the conjugate pad, a Cotinine test line 105, a CEMA test line 106 and a control test line 107. The skilled person will understand that the conjugate pad and the intermediate pad can be arranged as shown or swapped around, as required. The skilled person will also understand that the test lines can be arranged in any order, as required. The lateral flow device or dipstick 100 terminates with the absorption pad 108 to function as a waste container at the distal end. The conjugate pad 103 contains a porous material containing: (i) dried monoclonal tagged (for example, anti-FLAG-tag) antibody conjugated to a label (colloidal gold); and (ii) polyclonal anti-Cotinine antibody conjugated to the same label (colloidal gold). Different labels can be used if required. The intermediate pad 104 is made from a porous material containing dried anti-CEMA scFv with a tag (for example, a FLAG-Tag). A FLAG-tag, is a polypeptide tag that can be added to a protein which has the amino acid sequence motif DYKDDDDK (Hopp et al. (1988) Bio/Technology. 6 (10): 1204-10). The CEMA test line 106 contains the CEMA conjugate as described herein. The presence of the anti-CEMA scFv on the CEMA test line 106 is detected using the anti-Tag antibody conjugated to colloidal gold that was initially present in the conjugate pad. The Cotinine test line 105 contains a cotinine-protein conjugate. The presence of Cotinine is detected using the polyclonal anti-Cotinine antibody conjugated to the same label (colloidal gold) that was initially present in the conjugate pad. Although not shown, the device 100 can be housed in a liquid-tight or impervious housing allowing the immersion of the device 100 into a liquid sample. The housing can comprise or consist of plastic. The sample can be a liquid sample—such as urine. The device 100 can have an elongated shape, the dimensions of which can vary, depending on the actual use of the device; exemplary dimensions are from 6 to 8 cm in length, and 3 to 6 mm in width.

The results of an exemplary competitive immunoassay test format are shown in FIG. 10 in which a positive test for Cotinine and CEMA is indicative of the test subject being a smoker. In an embodiment of the invention, the limit of detection of the test is 200 ng/mL for the Cotinine test line and 160 ng/mL for the CEMA test line. When the amount of Cotinine in the test sample is greater than 200 ng/mL then a positive signal will be generated; when the amount of Cotinine in the test sample is less than 200 ng/mL then a negative signal will be generated. When the amount of CEMA in the test sample is greater than 160 ng/mL then a positive signal will be generated; when the amount of CEMA in the test sample is less than 160 ng/mL then a negative signal will be generated. In the competitive format, a positive result for Cotinine or CEMA will mean that the test line is absent and a negative result for Cotinine or CEMA will mean that the test line is present. A negative test for Cotinine and CEMA is indicative of the test subject being a non-smoker. A positive test for Cotinine and a negative test for CEMA is indicative of the test subject being a switcher from a smoker to a consumer of a RRP. The absence of a control line is indicative of an invalid test. A positive test indicates that the threshold of detection has been crossed. As the test configuration shown in FIG. 10 is a competitive assay, the result is positive when the CEMA or Cotinine test line is absent such that absence of a signal at a test line is an indication of the presence of the metabolite while appearance of signal indicates the absence of the metabolite. In certain embodiments, the competitive format is preferred for use in the present disclosure.

The disclosure further provides kits for the detection of one or more of the metabolites comprising means for detecting the level of the one or more metabolites in a sample from a subject. In a preferred embodiment, such a kit or kits are ideally designed for use at home or by a doctor in a general practice setting.

Further disclosed is a kit, particularly a kit for determining the smoking status of a subject as taught herein in a subject, the kit comprising (i) means for measuring the metabolites as taught herein, particularly in a sample from the subject, and (ii) optionally a reference value for the metabolite or metabolites or means for establishing said reference value, wherein said reference value represents detection of the metabolites.

A home-test kit may give the subject a readout that can be communicated to a medicinal practitioner, after which appropriate action can be taken. Non-limiting examples are: systems comprising specific binding molecules for the requisite metabolites(s) attached to a solid phase, for example, a portable lateral flow immunoassay device—such as a dipstick. One non-limiting example is to use a lateral flow test-strip and labelled antibody or antigen-binding fragment thereof which combination does not require any washing of a membrane. The lateral flow test strip is well known, for example, in the field of pregnancy testing kits where a first anti-hCG antibody is present on the support, and is carried complexed with hCG by the flow of urine onto an immobilised second anti-hCG antibody that permits visualisation. Other non-limiting examples of such home test devices, systems or kits may be found for example in the following U.S. Pat. Nos. 6,107,045, 6,974,706, 5,108,889, 6,027,944, 6,482,156, 6,511,814, 5,824,268, 5,726,010, 6,001,658 or U.S. patent applications: 2008/0090305 or 2003/0109067. There is also disclosed a kit for determining the smoking status of a subject comprising or consisting of: (i) a first device adapted to detect the presence of CEMA in a biological sample; and (ii) a second device adapted to detect the presence of Cotinine in a biological sample; and optionally, a set of instructions for determining the smoking status of the subject. The invention further provides a nucleic acid construct comprising a polynucleotide as described herein. Typically, the construct will be an expression vector allowing expression, in a suitable host, of the polypeptide(s) encoded by the polynucleotide. The construct may comprise, for example, one or more of the following: a promoter active in the host; one or more regulatory sequences, such as enhancers; an origin of replication; and a marker, preferably a selectable marker. The host may be a eukaryotic or prokaryotic host. The construct may comprise a polynucleotide which encodes a polypeptide encoding an scFv. The construct may comprise a polynucleotide which encodes a polypeptide comprising three light chain chains or three heavy chains. Alternatively, the polynucleotide may encode a polypeptide comprising three heavy chains and three light chains joined by a suitably flexible linker of appropriate length. Another possibility is that a single construct may comprise a polynucleotide encoding two separate polypeptides—one comprising the light chains and one comprising the heavy chains. The separate polypeptides may be independently expressed or may form part of a single common operon. The construct may comprise one or more regulatory features, such as an enhancer, an origin of replication, and one or more markers (selectable or otherwise). The construct may be provided in liquid or solid form, preferably as a freeze-dried powder which, typically, is rehydrated with a sterile aqueous liquid prior to use. A vector includes expression vectors and transformation vectors and shuttle vectors. An expression vector refers to a construct capable of in vivo or in vitro expression. A transformation vector is a construct capable of being transferred from one entity to another entity—which may be of the species or may be of a different species. If the construct is capable of being transferred from one species to another—such as from an Escherichia coli plasmid to a bacterium, such as of the genus Bacillus, then the transformation vector is sometimes called a shuttle vector. It may even be a construct capable of being transferred from an E. coli plasmid to an Agrobacterium to a plant.

Vectors may be transformed into a suitable host cell as described below to provide for expression of a polypeptide encompassed in the present invention. Thus, in a further aspect the invention provides a process for preparing polypeptides for use in the present invention which comprises cultivating a host cell transformed or transfected with an expression vector as described above under conditions to provide for expression by the vector of a coding sequence encoding the polypeptides, and recovering the expressed polypeptides. The vectors may be for example, plasmid, virus or phage vectors provided with an origin of replication, optionally a promoter for the expression of the said polynucleotide and optionally a regulator of the promoter. Vectors may contain one or more selectable marker genes which are well known in the art. There are many known heavy and light chain expression vectors commercially available. The skilled operator may choose vectors expressing the same constant region subtype as the original antibody. The sequence of the heavy and light chain variable regions is then easily placed into the vector accordingly. There are a wide range of known vectors commercially available for scFv expression.

The invention further provides a host cell—such as a host cell in vitro—comprising the polynucleotide or construct described herein. The host cell may be a bacterium, a yeast or other fungal cell, insect cell, a plant cell, or a mammalian cell, for example. In certain embodiments, the host cell is a bacterium.

E. coli is one prokaryotic host that may be of use and is preferred in certain embodiments. Other microbial hosts include bacilli, such as Bacillus subtilis, and other enterobacteriaceae, such as Salmonella, Serratia, and various Pseudomonas species. In these prokaryotic hosts, one can make expression vectors, which will typically contain expression control sequences compatible with the host cell (for example, an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Other microbes—such as yeast, may be used for expression. Saccharomyces is a preferred yeast host, with suitable vectors having expression control sequences (for example, promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

In addition to microorganisms, mammalian tissue cell culture may also be used to express and produce the antibodies or fragments thereof as described herein (see Winnacker, From Genes to Clones, VCH Publishers, N.Y., N.Y. (1987). A number of suitable host cell lines capable of secreting heterologous proteins (for example, intact immunoglobulins) have been developed in the art, and include CHO cell lines, various Cos cell lines, HeLa cells, myeloma cell lines, or transformed B-cells or hybridomas. CHO cells are preferred in certain embodiments. Alternatively, antibody-coding sequences can be incorporated into transgenes for introduction into the genome of a transgenic animal and subsequent expression in the milk of the transgenic animal (see, for example, U.S. Pat. No. 5,741,957). Suitable transgenes include coding sequences for light and/or heavy chains in operable linkage with a promoter and enhancer from a mammary gland specific gene, such as casein or beta lactoglobulin.

Alternatively, the antibodies or antigen-binding fragments thereof described herein can be produced in transgenic plants—such as tobacco, maize, soybean and alfalfa. Improved ‘plantibody’ vectors (see Hendy et al. (1999) J. Immunol. Methods 231:137-146) and purification strategies coupled with an increase in transformable crop species render such methods a practical and efficient means of producing recombinant immunoglobulins. Moreover, plant produced antibodies have been shown to be safe and effective.

Full length antibody or antigen-binding fragment or antibody fusion proteins can be produced in bacteria. Production in E. coli is faster and more cost efficient. For expression of antigen-binding fragment and polypeptides in bacteria, see, for example, U.S. Pat. Nos. 5,648,237, 5,789,199 and 5,840,523.

The invention is further described in the Examples below, which are provided to describe the invention in further detail. These examples, which set forth a preferred mode presently contemplated for carrying out the invention, are intended to illustrate and not to limit the invention.

### EXAMPLES

### Example 1—Synthesis of CEMA Linked Conjugates

Cyanoethylmercapturic acid (CEMA) is conjugated to beta-thyroglobulin (BTG) as a carrier protein using disuccinimidyl suberate (DSS) or ethylene glycol bis(succinimidyl succinate) (EGS) linkers. First, CEMA and BTG are diluted to 20 mg/ml in PBS (pH 7.4: 137 mM NaCl, 2.7 mM KCl, 10 mM Na₂HPO₄, 1.8 mM KH₂PO₄ dissolved in water). EGS or DSS are separately dissolved in dry DMSO to 15 mM for 2 hours at room temperature before centrifugation for 2 minutes at 2000 rpm, and the resulting supernatant collected. The supernatant is then added at 10× molar excess to the CEMA-BTG mixture and incubated for four hours at room temperature before quenching with 1 M Tris buffer (pH 7.4) for 30 minutes at a final concentration of 20 mM. The mixture is then centrifuged for 2 minutes at 2000 rpm, and the supernatant collected before dialysing in Tris/Borate/EDTA (TBE) buffer (10× buffer: 1 M Tris base, 1 M Boric acid, 0.02 M EDTA in RNase-free water, diluted to 1× for use) for 2 hours to remove the remaining DMSO.

### Example 2—Immunisation and Anti-CEMA Library Construction

3-month-old New Zealand rabbits are injected subcutaneously with 1 mg BTG-DSS-CEMA or BTG-EGS-CEMA immunogens (prepared according to Example 1) and diluted in Incomplete Freund's Adjuvant in a 1:1 ratio. Rabbits are injected every two weeks, with up to five injections total. Antibody titers are tested by obtaining 5 mL of blood sample sera from the ear vessels one week after the third to fifth injections. Blood is then allowed to clot for one hour at room temperature before centrifugation at 3200 rpm for 15 minutes and the serum layer pipetted into a new tube. The antibody titer of the collected serum is then tested using an indirect ELISA assay against BSA-DSS-CEMA, BSA-EGS-CEMA and BSA as negative control. To do this, 96-well plates are coated with 1 μg/ml conjugated antigens (in PBS, pH 7.5) overnight at 4° C. Plates are then washed three times with PBS before blocking with 2.5% Milk in PBS (pH 7.4) and washed a further three times with PBS. 50 μL of the primary serum (diluted 1:100 and serially diluted 1/10) is then added per well and plates incubated for one hour at 37° C. before washing three times with PBS. 50 μL anti-rabbit IgG antibody conjugated to HRP (Jackson 111-036-046) is then added at 1/5000 dilution and incubated for one hour at 37° C. before washing again three times with PBS. Finally, wells are incubated with 50 μL TMB (KPL 52-00-01) for 10 minutes before stopping by adding 50 μL acid stop solution (H₂SO₄ 0.1 M). The signal is then measured at 450 nm in a BioteK EL808 reader.

The titer of the response is calculated as the dilution giving 50% of the highest signal in ELISA. Rabbits with the highest antibody titer (1/170000) are selected and injected one further time with 1 mg BTG-DSS-CEMA or BTG-EGS-CEMA diluted in Incomplete Freund's Adjuvant in a 1:1 ratio before euthanizing and removing the spleen.

Spleen is harvested and cut into small pieces in Trizol reagent and crushed using a dispersing instrument (IKA T18D Ultra-Turrax) and mRNA is then extracted by the standard TrizoVBCP protocol. To do this, the crushed rabbit spleen in 30 ml of TRI reagent is incubated for 5 minutes at room temperature and then centrifuged (4° C., 2500 g, 10 minutes), then 3 mL of BCP is added to the supernatant in new tubes and incubated for 15 minutes at room temperature. The tube is centrifuged (4° C., 17500 g, 15 minutes), and 12 mL isopropanol is added to the supernatant, vortexed (15 seconds) and incubated for 10 minutes at room temperature. Then, after centrifugation (4° C., 17500 g, 10 minutes) the pellet of total RNA is washed using 1.5 mL ethanol 75%, centrifuged (4° C., 17500 g, 10 minutes), and then dried at RT. For quantification, the RNA pellet is dissolved in molecular biology grade water and quantified on a spectrometer (Nanodrop). Then, the total RNA is analyzed on 0.8% (w/v) agarose gel, thereby confirming 18S and 28S rRNA bands and the remaining RNA is stored at a temperature of −20° C.

For immune library construction, 20 μg total RNA is reverse transcribed using the SuperScript III first-strand synthesis (SuperMix (Invitrogen)) according to the manufacturer's instructions. In a first step, 20 μg of the isolated mRNA is mixed with 1 μL annealing buffer and 1 μL (50 μM) oligo dT, incubated for 5 minutes at 65° C. and placed on ice for 1 minute. Subsequently, while still on ice, 10 μl 2× First-Strand Reaction Mix and SuperScript™ III (Thermo Fisher Scientific)/RNaseOUT™ Enzyme Mix (Thermo Fisher Scientific) is added following the manufacturer's instructions. After mixing, the reaction is placed for 50 minutes at 50° C. The reaction is terminated by incubation at 85° C. for 5 minutes. After termination, the reaction mix is placed on ice or stored at −20° C. in case of future need, as cDNA is more stable than RNA for storage. A PCR is performed to amplify the cDNA encoding variable domains VLκ, VLλ and VH separately using the rabbit specific primers VLκ, VLλ and VH forward and reverse primers (Ridder, R., Schmitz, R., Legay, F. et al. Generation of Rabbit Monoclonal Antibody Fragments from a Combinatorial Phage Display Library and Their Production in the Yeast Pichia pastoris. Nat Biotechnol 13, 255-260 (1995)). For a 50 μL total volume PCR reaction, 2 μL cDNA product, 10×PCR buffer (100 mM Tris-HCl, 15 mM MgCl₂, and 50 mM KCl, pH 8.3), 0.2 μM primers, 0.2 mM dNTPs, 1 mM MgCl₂, and 5 units of Taq DNA polymerase are introduced into a reaction vessel and a final volume of a reaction mixture is adjusted by sterile triple-distilled water. The PCR reaction consists of 35 cycles, each cycle including: heating to denature DNA at 94° C. for 5 minutes (pre-denaturation), 94° C. for 30 seconds (denaturation), 55° C. for 30 seconds (annealing) and 72° C. for 60 seconds (polymerization). After the final cycle, the PCR reaction is terminated by heating at 72° C. for another 10 minutes. After PCR, 5 μL of each PCR product is analysed on an agarose gel (0.8%) and only VLκ, VLλ and VH reactions showing amplification are pooled.

The cDNA fragments encoding the VL (VLκ and VLλ) and the VH, respectively, are electrophoresed on a 1.5% agarose gel and extracted/purified using a gel extraction kit (Illustra GFXR, GE) following manufacturer instructions and cloned in a pGemT vector (Promega) as backup material, with ligation reactions prepared as follows:

| | | | | Lambda |
|---|---|---|---|---|
| | Negative | Fd | Kappa | ligation |
| μL | control | ligation | ligation | (optional) |
| Buffer 2x | 5 | 5 | 5 | 5 |
| pGemT | 1 | 1 | 1 | 1 |
| PCR | — | 3 μL of | 3 μL of | 3 μL of |
| products | | pooled | pooled | pooled |
| | | H reactions | Kappa | Lambda |
| | | | reactions | reactions |
| Ligase | 1 | 1 | 1 | 1 |
| Water | 3 | — | — | — |

The 3 ligation reactions are incubated at 4° C., overnight and stored at −20° C.

The PCR products of the heavy chain and light chain are cloned sequentially in the pTH1 phagemid vector (Biotem, France) as follows.

In a first step, the pTH1 and VL domain vectors for library cloning are prepared. Both vectors are digested for VL cloning as follows:

| | | |
|---|---|---|
| Solution or component | Volume | Final concentration |

| | | | |
|---|---|---|---|
| dH2O | 83-x | μL | — |
| pHAL14 or VL | x | μL | 5 μg or 2 μg |
| NEB buffer 3 (10x) | 10 | μL | 1x |
| BSA (100x) | 1 | μL | 1x |
| NEB Mlul (10 U/μl) | 3 | μL | 30 U |
| NEB Notl (10 U/μl) | 3 | μL | |

The digestion is incubated at 37° C. for 2 h and controlled on 1.5% TAE agarose gel electrophoresis. The enzymes is inactivated at 65° C. for 10 min, and 0.5 μL CIP (1 U/μL) is added and incubated at 37° C. for 30 minutes. Both pTH1 and VL domains are purified using a PCR purification kit according to the manufacturers instructions and eluted with 50 μL elution buffer or water.

The VL (˜380 bp) is used for ligation in the vector pTH1 as follows:

| | | |
|---|---|---|
| Solution or component | Volume | Final concentration |

| dH₂O | 89-x-y | μL | — | |
|---|---|---|---|---|
| PHAL14 | x | μL | 1000 | ng |
| VL (kappa or lambda) | y | μL | 270 | ng |
| T4 ligase buffer (10x) | 10 | μL | 1x | |
| T4 ligase (3 U/μL) | 1 | μL | 3 | U |

incubated at 16° C. overnight and the ligation product is precipitated with 10 μL 3 M sodium acetate pH 5.2 and 250 μL ethanol, incubated for 2 minutes at room temperature and centrifuged for 5 minutes at 16000 g @ 4° C. The pellet is washed with ethanol and centrifuged for 2 minutes at 16000 g @4° C. before being resolved in 35 μL dH2O. For DNA amplification, the ligation reaction is incubated with electrocompetent XL1-Blue MRF′ cells, before electroporation using 1.7 kV pulse. The pulse time is between 4-5 ms for optimal electroporation efficiency. Immediately, 1 mL 37° C. pre-warmed SOC medium is added, and stored in a 2 mL cap and shaken for 1 h at 600 rpm and 37° C. To determine the amount of transformants, 10 μL (=10⁻² dilution) of the transformation is used to perform a dilution series down to 10⁻⁶ dilution and plated out a 10⁻⁶ dilution on 2xYT-GAT agar plates, incubated overnight at 37° C. The remaining 990 μL is plated on 2xYT-GAT agar plates and incubated overnight at 37° C. Then, the colonies on the plate are harvested with 40 mL 2xYT medium using a drigalsky spatula, and 5 mL bacteria solution is used for midi plasmid preparation according to the manufacturer's instructions.

For the second cloning step, the pTH1-VL and pGemT-VH repertoire are digested as follows:

| | | |
|---|---|---|
| Solution or component | Volume | Final concentration |

| | | | |
|---|---|---|---|
| dH₂O | 81-x | μL | — |
| pTH1-VL or VH | x | μL | 5 μg or 2 μg |
| NEB buffer 2 (10x) | 10 | μL | 1x |
| BSA (100x) | 1 | μL | 1x |
| NEB Ncol (10 U/μl) | 3 | μL | 30 U |
| NEB HindIII (20 U/μl) | 5 | μL | 100 U |

The digestion is incubated at 37° C. for 2 h and controlled on 1.5% TAE agarose gel electrophoresis. The enzymes are inactivated at 65° C. for 10 min, and 0.5 μL CIP (1 U/μL) is added and incubated at 37° C. for 30 minutes. Both pTH1-VL and VH domains are purified using a PCR purification Kit according the manufacturer's instructions and eluted with 50 μL elution buffer or water. The vector pTH1-VL (˜4610 bp) and VH (˜380 bp) is ligated as follows:

| | | |
|---|---|---|
| Solution or component | Volume | Final concentration |

| dH₂O | 89-x-y | μL | — | |
|---|---|---|---|---|
| pTH1 | x | μL | 1000 | ng |
| VH | y | μL | 250 | ng |
| T4 ligase buffer (10x) | 10 | μL | 1x | |
| T4 ligase (3 U/μL) | 1 | μL | 3 | U |

incubated at 16° C. overnight and the ligation product is precipitated with 10 μL 3 M sodium acetate pH 5.2 and 250 μL ethanol, incubated for 2 minutes at room temperature and centrifuged for 5 minutes at 16000 g @ 4° C. The pellet is washed with ethanol and centrifuged for 2 minutes at 16000 g @ 4° C. before being resolved in 35 μL dH2O. For DNA amplification, the ligation reaction is incubated with electrocompetent XL1-Blue MRF′ cells, before electroporation using 1.7 kV pulse. The pulse time is between 4-5 ms for optimal electroporation efficiency. Immediately, 1 mL 37° C. pre-warmed SOC medium is added, and stored to a 2 mL cap and shaken for 1 hour at 600 rpm and 37° C. To determine the amount of transformants, 10 μL (=10−2 dilution) of the transformation is used to perform a dilution series down to 10⁻⁶ dilution and plated out a 10⁻⁶ dilution on 2xYT-GAT agar plates and incubated overnight at 37° C. The remaining 990 μL is plated on 2xYT-GAT agar plates and incubated overnight at 37° C. Then, the colonies on the plate are harvested with 40 mL 2xYT medium using a drigalsky spatula, and 5 mL bacteria solution is used for midi plasmid preparation according to the manufacturer's instructions and 800 μL bacteria solution is used for glycerol stocks. 5-25 glycerol stocks are made per sub-library and stored at −80° C.

To package the library, 400 mL 2xTY-GA in a 1 L Erlenmeyer flask is inoculated with 1 mL antibody gene library stock and incubated at 250 rpm at 37° C. up to an O.D.600 nm˜0.5. The bacteria culture (˜1.25*10¹⁰ cells) is mixed with 2.5*10¹¹ colony forming units (cfu) of the helper phage M13K07 and incubated for 30 minutes without shaking and the following 30 minutes with 250 rpm at 37° C. scFv-phage library is produced overnight at 250 rpm @ 30° C. The phage is harvested from the supernatant by adding ⅕ volume PEG solution, incubated for 1 hour at 4° C. with gentle shaking, followed by centrifugation for 1 hour at 10000 g. The supernatant is discarded, and the phage pellet suspended in 10 mL phage dilution buffer and ⅕ volume PEG solution added, incubated on ice for 20 minutes and pelleted by centrifugation for 30 minutes at 10000 g. Then the supernatant is discarded and the phage pellet suspended in 1 mL phage dilution TBS buffer.

### Example 3—Panning

For selection of phage displayed antibody fragments, panning is performed. To do this, 96-well Nunc Maxisorp round-bottom microtiter plates (Thermo Fisher Scientific) are coated overnight at 4° C. with 4 mL of 10-100 μg/mL antigen solution (BTG-EGS-CEMA, BTG-DSS-CEMA in 0.1 M NaHCO₃ buffer, pH 8.6). The coated wells are washed and blocked with 300 μL PBS containing 4% skimmed milk (4% PBSM) and washed twice with PBS/0.1% Tween 20 (Sigma) and twice with PBS. Then, 100 μL of the freshly prepared phages containing 7×10⁷ phages are added to each well and the plate is incubated at 37° C. for 2 hours with gentle rocking. Each well is washed five times with 200 μL PBS containing 0.5% Tween 20. The bound phages in each well are eluted using 0.05% trypsin (Thermo Fisher Scientific). The eluted phages are then reamplified by transformation of E. coli(XL1 blue MRF′) and incubation with M13K07 helper phage (as described above) and titered. The new phages are used for further panning until significant enrichment of the CEMA specific phage is achieved. In total, seven biopanning rounds are carried out against CEMA to enrich the binding phages.

### Example 4—Identification of scFv with Strongest Binding

After panning, specific antigen binding is determined by ELISA to identify the Phages/scFv with strongest binding. To do this, ELISA-Phage is performed by coating 96-well plates with BTG-EGS-CEMA or BTG-DSS-CEMA, diluted in PBS (pH 7.5) overnight at 4° C. Plates are then washed three times with PBS before blocking with 1% BSA in Tris-buffered (15 mM) saline (TBS, pH 7.4) and washed a further three times with PBS. 50 μL of the selected colony is then added per well and plates are incubated for one hour at 37° C. before washing three times with PBS. Secondary, 50 μL anti-M13KO7 antibody conjugated to HRP is then added at dilution and incubated for one hour at 37° C. before washing again three times with PBS. Finally, wells are incubated with 50 μL TMB substrate for 5 minutes before stopping by adding 50 μL acid stop solution. The signals are measured at 450 nm in a BioteK EL808 reader. The twenty-eight antibodies with the highest BTG-EGS-CEMA/BTG (signal/background) ratio are regarded as positives. DNA extraction is performed on these twenty-eight clones and eight, non-redundant sequences identified by sequence alignment. These eight scFv are then produced as soluble scFv and tested in ELISA and LFIA.

### Example 5—Production of the Selected scFv and Comparison of the Sensitivity Using EGS or DSS in ELISA

For soluble scFv production, clone DNA is used for E. coli HB2151 strain transformation, an E. coli strain dedicated to production. The scFv, expressed as soluble scFv, is purified using the HIS-Tag on NI-NTA column (Qiagen), according to manufacturer's instructions.

In competitive ELISAs, the non-labelled, primary scFv is incubated with the target antigen. To do this, a 96-well ELISA plate is precoated with a 1 μg/ml BTG-EGS-CEMA or BTG-DSS-CEMA coating overnight at 4° C. and washed the next day three times, and incubated with a blocking buffer (PBS, pH 7.4 containing milk 2.5%) for 1 hour at 37° C. to reduce non-specific binding. Then, non-labelled, primary scFv R4C-B11, R5C-G4, R5C-F8, R5C-G6, R5C-G9 and R5C-E6 are added to the scFv-CEMA mixture wells of the pre-coated ELISA plate, and incubated at 37° C. for 1 hour. Wells are then washed three times with PBS, before incubation with anti-Flag secondary antibody conjugated to HRP at 37° C. for another hour at 37° C. before washing again with PBS and incubating with TMB substrate for 5 minutes. The reaction is then stopped by adding stop solution and signal is measured at 450 nm for light absorbance (O.D. value). The standard curve is generated using the same method but instead of adding samples, a dilution series of recombinant CEMA with known concentrations is added to 6-8 wells. The O.D. is then used to calculate the amount of molecule of interest in each well, by comparing each sample well against the standard curve.

As demonstrated in FIG. 2, R4C—B11, R5C-G4, R5C-F8, R5C-G6, and R5C-E6 had high percentage inhibition of 77-97% in the presence of 10 μg/mL free CEMA. However, at 1 μg/mL free CEMA it is possible to identify the scFv with the best sensitivity. The most sensitive scFv for BTG-EGS-CEMA are as follows: R4C—B11>R5C-F8>R5C-G6>R5C-E6=R5C-G4. Similarly. FIG. 3 demonstrates that R4C—B11, R5C-G4, R5C-F8, and R5C-E6 are effective at inhibiting binding of BTG-DSS-CEMA in the presence of 10 μg/mL free CEMA. At 0.1 μg/mL free CEMA, however, the percentage inhibition indicates that the scFv with the best sensitivity for BTG-DSS-CEMA is as follows: R4C—B11>R5C-G6>R5C-F8>R5C-E6>R5C-G4.

### Example 6—Comparison of the Sensitivity of Selected scFv Prepared Using EGS or DSS in Lateral Flow Immunoassay

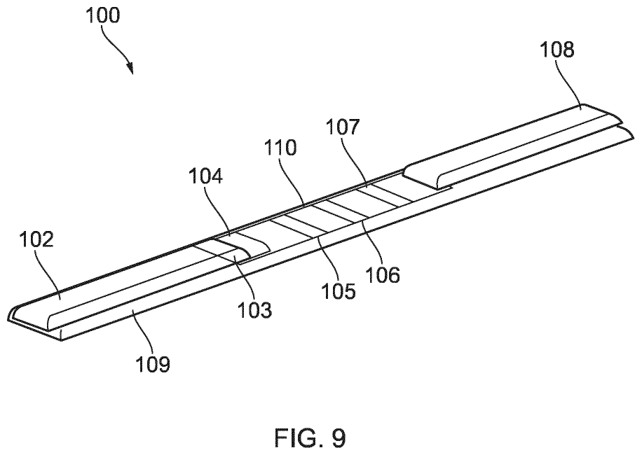

In an exemplary lateral flow immunoassay, the lateral flow test strip comprises (i) a sample pad; (ii) a conjugate pad; (iii) an intermediate pad, (iv) a detection zone; and (v) an optional absorption pad (see FIG. 9). The conjugate pad (cellulose fibre) is prepared by saturating the pad in Hepes (pH 8) with Tween 20, 0.5% BSA (weight/volume) and a dilution at an optical density at 530 nm (OD) of 5 of monoclonal anti-FLAG-tag antibody conjugated to colloidal gold and a dilution at an OD 0.5 of polyclonal anti-Cotinine antibody conjugated to colloidal gold. The pad is dried overnight at 37° C. The intermediate pad is prepared by soaking the pad in Hepes (pH 8) with Tween 20, 0.5% BSA (weight/volume) and a 10 μg/ml dilution of anti-CEMA scFv. The detection zones comprise two test lines with microsprayed 0.25 mg/ml free BSA-DSS-CEMA or BSA-EGS-CEMA or 0.25 mg/ml free cotinine-BSA onto a nitrocellulose membrane and the membrane is dried overnight at 37° C. Therefore, when the sample pad of the lateral flow test strip is dipped into the sample liquid (for example, urine) or when the sample is added into the sample well of the cassette using a pipette, the urine is drawn through the strip by capillary action through each pad, freeing the dried anti-Cotinine and anti-FLAG-tag antibodies into the intermediate pad, where the anti-CEMA scFv binds to CEMA in the sample, and the anti-FLAG-tag antibody binds to the anti-CEMA scFv. Similarly, the anti-Cotinine antibody binds to Cotinine in the urine. These immunogen-antibody complexes are then further drawn up the lateral flow test strip onto the test lines where antibodies bound to Cotinine or CEMA in the urine will be unable to bind to the test line Cotinine or CEMA, and thus produces no line. However, if the urine sample is negative then the anti-CEMA and anti-Cotinine antibodies are free to bind to the Cotinine and CEMA on the test lines, producing a coloured line. The test result is read at 5 minutes after addition of the urine sample, and there is a control line consisting of goat anti-mouse antibodies (see FIGS. 9 and 10).

When performing LFIA, lateral flow test strips are prepared using each of the eight anti-CEMA scFvs described in Example 4 on the intermediate pad.

As shown in FIG. 4, the scFv are slightly better at binding to the BTG-EGS-CEMA immunogen than the BTG-DSS-CEMA immunogen. Similarly, FIG. 4 indicates that scFV R5C-G12 and R5C-G9 do not recognize both immunogens. scFV R5C-G8 also seems to have low recognition of CEMA.

### Example 7—Inhibition Profile in Urine

As scFV R5C-G12, R5C-G9 and R5C-G8 show poor reactivity in Example 6, only R4C—B11, R5C-G4, R5C-F8, R5C-G6, and R5C-E6 are used to determine the inhibition profile of urine in the lateral flow immunoassay test in this Example.

The lateral flow immunoassay test is performed (as described in Example 6) using urine containing 10-500 ng/mL CEMA, with 1×PBS and CEMA negative urine as negative controls on the BTG-EGS-CEMA coating on the test line. As demonstrated in FIG. 5, scFv R5C-G6 and R5C-F8 are slightly inhibited in the presence of negative urine and therefore may not be suitable for use in urine lateral flow tests as there would be a risk of false positives. The inhibition is believed to be due to the high ionic content of urine.

However, as shown in FIG. 5, scFv R4C—B11, R5C-E6 and RSC-G4 show a negative result for both the 1×PBS and negative urine samples, but show strong inhibition in the presence of 500 ng/mL and 100 ng/mL CEMA in the urine samples. R4C—B11 also shows strong inhibition in the 50 ng/mL CEMA urine sample. As shown in FIG. 5, scFv R4C—B11, R5C-E6 and ESC-G4 all show a negative response in the presence of very small amounts of CEMA in the urine samples (10-20 ng/mL).

Therefore, only scFv R5C-G4, R4C—B11, R5C-E6 are used for performing a lateral flow immunoassay on the BTG-DSS-CEMA coating (see FIG. 6). Each of the three selected scFvs demonstrate good inhibition in the 50-500 ng/mL CEMA urine samples. scFv R5C-G4, R4C—B11, R5C-E6 are therefore assayed in dilutions from 0.488-535 ng/mL CEMA (FIG. 7). Each of scFv R5C-G4, R4C—B11, R5C-E6 allow detection of positive urine samples which contain over 100 ng/mL. Samples with 35 ng/mL or under appear as negative.

### Example 8—Specificity of Selected scFv

Based on the results of Examples 5, 6 and 7, the specificity of scFv R5C-G4, R4C—B11, and R5C-E6 is tested using lateral flow immunoassay with other molecules on the test line that have a structure similar to that of CEMA, such as HEMA, MHBMA, 3-HPMA, DHBMA at a concentration of 1 μg/mL.

As demonstrated in FIG. 8, there is no cross-reactivity of scFv R5C-G4, R4C—B11, and R5C-E6 with any of the similar molecules. Therefore, scFv R5C-G4, R4C—B11, and R5C-E6 are highly specific to CEMA.

### Example 9—Rapid Test Kit for the Qualitative Detection of CEMA and Cotinine in Human Urine

A rapid lateral flow immunochromatographic assay test kit is devised for the qualitative detection of Cotinine and CEMA in human urine. The test kit aims to distinguish cigarette smokers, RRP (Reduced Risk Product) users and non-smokers.

The kit is a competitive immunochromatographic competitive assay for the detection of Cotinine and CEMA in human urines. When CEMA and/or Cotinine are absent in the sample, the test lines appear, but when CEMA or Cotinine are present in the sample, the signal is inhibited and the test lines do not appear. During the test, the urine sample reacts with colloidal gold nanoparticles conjugated to either polyclonal antibodies directed against Cotinine or monoclonal antibodies or antigen binding fragments thereof directed against CEMA. Then, the mixture migrates through the membrane by capillary action, and meet Cotinine-BSA and CEMA-BSA conjugate molecules printed respectively on the test lines 1 and 2. If Cotinine and/or CEMA is present in the sample, they will inhibit the binding of the antibodies, preventing a coloured test line to appear. Inversely, if the sample is free of Cotinine and/or CEMA, the conjugated antibodies will recognize the CEMA or Cotinine conjugate molecules on the test lines leading to the appearance of a test line. A coloured line should always be present on the Control line marked “C”, indicating that a sufficient volume or urine was applied and that the test migration occurred property.

Specimen collection: Collect the urine in the provided collection cup and identify the sample by writing on the side the name of the donor and the date of the urine collection. With the provided pipette, collect 100 μL of urine and dispense the total volume of the liquid contained within the pipette tube in the sample well of the device by pressing on the top of the reservoir. Read the result between 5 and 10 minutes. Do not read after 10 minutes.

Negative result: Three lines appear, one in the control area (C) and one in each test lines area Cotinine (1) and CEMA (2). It should be interpreted as a non-smoker urine. A negative result can also be obtained if the concentration of Cotinine and CEMA is lower than the limit of detection (low number of cigarettes per day).

Positive result: One line appears within the control area (C). It should be interpreted as a smoker urine (both CEMA and Cotinine in the urine). Two lines appear within the control area (C) and the CEMA test line (2). It should be interpreted as a switcher urine (only Cotinine in the urine).

Invalid result: If the control line (C) does not appear, the test result is invalid. Repeat the procedure using a new test.

Further aspects of the present disclosure are set forth below in the following numbered paragraphs:
1. An antibody or antigen binding fragment thereof: (i) capable of binding to N-acetyl-S-[2-carboxyethyl]-L-cysteine (CEMA) and; (ii) capable of binding to a conjugate comprising a compound of formula [II]:

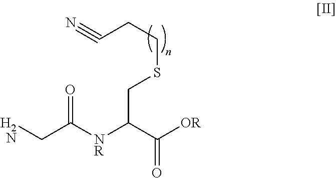

wherein n is selected from 0 to 4 (that is, 0, 1, 2, 3, or 4), and each R is independently selected from H or C₁ to C₆ alkyl; preferably, a compound of formula [I]:

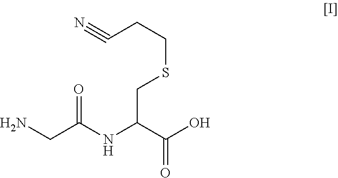 [SMILES: N#CCCSCC(NC(=O)CN)C(=O)O]

wherein the compound of formula [II] or formula [I] is coupled to an immunogenic carrier via a linker, suitably, wherein the linker is coupled to the compound of formula [I] via the amine group.
2. The antibody or antigen binding fragment thereof according to paragraph 1, wherein the immunogenic carrier is a protein, preferably, bovine serum albumin or bovine thyroglobulin.
3. The antibody or antigen binding fragment thereof according to paragraph 1 or paragraph 2, wherein the linker is glycol bis(succinimidyl succinate) (EGS) or disuccinimidyl suberate (DSS).
4. The antibody or antigen binding fragment thereof according to any of the preceding paragraphs, wherein the antibody is a monoclonal antibody, preferably wherein the antigen binding fragment thereof is a Fab fragment, a Fab′ fragment, a F(ab′)2 fragment, a scFv, a Fv, a rIgG, or a diabody, more preferably a scFv.
5. The antibody or antigen binding fragment thereof according to any of the preceding paragraphs, wherein the antibody does not bind to Cotinine or 2-hydroxyethylmethacrylate (HEMA) or monohydroxybutenyl-mercapturic acid (MHBMA) or 3-hydroxypropylmercapturic acid (3-HPMA) or dihydroxybutyl mercapturic acid (DHBMA).
6. The antibody or antigen binding fragment thereof according to any of the preceding paragraphs, wherein the percentage inhibition by urine is 20% or less, 10% or less, 5% or less or no inhibition.
7. The antibody or antigen binding fragment thereof according to any of the preceding paragraphs, wherein the antibody or antigen binding fragment thereof has a limit of detection for CEMA of 160 ng/mL CEMA in urine in an immunoassay.
8. An antibody or antigen binding fragment thereof comprising: VH CDR1, VH CDR2 and VH CDR3 consisting of the amino acid sequences of SEQ ID NO:3, SEQ ID NO:4 and SEQ ID NO:5 respectively, and VL CDR1, VL CDR2 and VL CDR3 consisting of the amino acid sequences of SEQ ID NO:7, SEQ ID NO:8 and SEQ ID NO:9 respectively; or VH CDR1, VH CDR2 and VH CDR3 consisting of the amino acid sequences of SEQ ID NO:12, SEQ ID NO:13 and SEQ ID NO:14 respectively, and VL CDR1, VL CDR2 and VL CDR3 consisting of the amino acid sequences of SEQ ID NO:16, SEQ ID NO:17 and SEQ ID NO:18 respectively; or VH CDR1, VH CDR2 and VH CDR3 consisting of the amino acid sequences of SEQ ID NO:21, SEQ ID NO:22 and SEQ ID NO:23 respectively, and VL CDR1, VL CDR2 and VL CDR3 consisting of the amino acid sequences of SEQ ID NO:25, SEQ ID NO:26 and SEQ ID NO:27 respectively.
9. The antibody or antigen binding fragment according to paragraph 8, wherein the antibody or antigen binding fragment thereof comprises VH amino acid sequence consisting of the amino acid sequence of SEQ ID NO: 2 or 11 or 20.
10. The antibody or antigen binding fragment according to paragraph 8 or paragraph 9, wherein the antibody or antigen binding fragment thereof comprises VL amino acid sequence consisting of the amino acid sequence of SEQ ID NO:6 or 15 or 24.
11. The antibody or antigen binding fragment according to paragraph 8, wherein the antibody or antigen binding fragment thereof comprises VH amino acid sequence consisting of the amino acid sequence of SEQ ID NO: 2 and VL amino acid sequence consisting of the amino acid sequence of SEQ ID NO:6; or wherein said antibody or antigen binding fragment thereof comprises VH amino acid sequence consisting of the amino acid sequence of SEQ ID NO:11 and VL amino acid sequence consisting of the amino acid sequence of SEQ ID NO:15; or wherein said antibody or antigen binding fragment thereof comprises VH amino acid sequence consisting of the amino acid sequence of SEQ ID NO:20 and VL amino acid sequence consisting of the amino acid sequence of SEQ ID NO:24.
12. The antibody or antigen binding fragment thereof according to any of paragraphs 8 to 12, wherein the antigen binding fragment thereof is selected from the group consisting of a Fab fragment, a Fab′ fragment, a F(ab)₂ fragment, a scFv, a Fv, a rIgG, and a diabody, preferably scFv.
13. The antibody or antigen binding fragment thereof according to paragraph 12, wherein the antigen binding fragment is a scFv and wherein said scFv comprises the amino acid sequence of SEQ ID NO: 1 or SEQ ID NO: 10 or SEQ ID NO: 19.
14. A polynucleotide encoding the antibody or antigen binding fragment thereof according to any of paragraphs 1 to 13 or a polynucleotide complementary thereto.
15. The polynucleotide according to paragraph 14 comprising one or more polynucleotide sequences selected from the group consisting of SEQ ID NO:28, SEQ ID NO: 31 and SEQ ID NO:34 or a polynucleotide complementary thereto.
16. A vector comprising the polynucleotide sequence according to paragraph 14 or paragraph 15.
17. The vector according to paragraph 16, further comprising an expression control sequence operatively linked to the nucleic acid encoding the variable heavy chain domain and/or the variable light chain domain.
18. A host cell containing the vector according to paragraph 16 or paragraph 17.
19. The host cell according to paragraph 18, wherein the host cell is a eukaryotic cell or a prokaryotic cell.
20. The host cell according to paragraph 19, wherein the eukaryotic cell is a Chinese Hamster Ovary (CHO) cell.
21. The host cell according to paragraph 19, wherein the prokaryotic cell is an E. coli cell.
22. A method of producing an antibody or antigen binding fragment thereof, comprising incubating the host cell of any of paragraphs 18 to 20 such that the encoded variable heavy chain domain and/or variable light chain domain is expressed by the cell; and recovering the expressed antibody or antigen binding fragment thereof.
23. The method according to paragraph 22, further comprising isolating and/or purifying the recovered antibody or antigen binding fragment thereof.
24. A method for producing the antibody of any of paragraphs 1 to 13, comprising immunising a non-human animal with a conjugate comprising a compound of formula [II]:

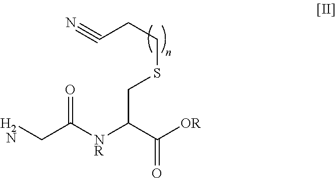

wherein n is selected from 0 to 4 (that is, 0, 1, 2, 3, or 4), and each R is independently selected from H or C₁ to C₆ alkyl; preferably, a compound of formula [I]:

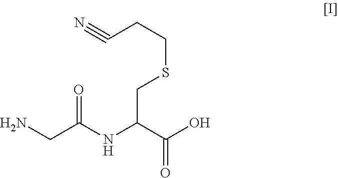 [SMILES: N#CCCSCC(NC(=O)CN)C(=O)O]

wherein said compound of formula [II] or formula [I] is coupled to an immunogenic carrier via a linker, suitably, wherein the linker is coupled to the compound of formula [I] via the amine group.
25. A device for determining the presence or absence of CEMA in a sample, said device comprising the antibody or antigen binding fragment thereof according to any of paragraphs 1 to 13 immobilised on a solid phase of the device.
26. The device according to paragraph 25, wherein the device is a portable lateral flow immunoassay device, preferably, a dipstick.
27. The device according to paragraph 25 or paragraph 26, wherein the device comprises: (i) a sample pad for receiving a sample; (ii) a conjugate pad in fluid communication with the sample pad; (iii) at least one detection zone in fluid communication with a distal end of the conjugate pad; and (iv) an adsorbent pad in fluid communication with a distal end of the detection zone.
28. The device according to paragraph 27, wherein the conjugate pad comprises the antibody or antigen binding fragment thereof according to any of paragraphs 1 to 13, wherein said antibody or antigen binding fragment thereof is labelled; and optionally a labelled antibody or antigen binding fragment thereof capable of binding cotinine.
29. The device according to paragraph 28, wherein the labelled antibody or antigen binding fragment thereof according to any of paragraphs 1 to 13 and labelled antibody or antigen binding fragment thereof capable of binding Cotinine are contained in separate pads in fluid communication with each other.
30. The device according to paragraph 28, wherein the labelled antibody or antigen binding fragment thereof is separately contained in an intermediate pad of the device that is located adjacent the conjugate pad and optionally, wherein, a labelled antibody or antigen binding fragment thereof capable of binding Cotinine is separately contained in the conjugate pad.
31. The device according to any of paragraphs 25 to 30, wherein the detection zone comprises CEMA and optionally Cotinine immobilised thereon, suitably, wherein the CEMA is in the form of the conjugate according to paragraph 1 or paragraph 2.
32. A method for detecting CEMA in a sample comprising the use of the device according to any of paragraphs 25 to 31 or a method for detecting CEMA and Cotinine in a sample comprising the use of the device according to any of paragraphs 25 to 31.
33. The method according to paragraph 32, comprising: (i) applying an aliquot of a liquid biological sample, preferably urine, to the sample pad, whereby the liquid biological sample is transferred by capillary action along a flow path defined by the sample pad, the conjugate pad, the detection zone; and the adsorbent pad; and (ii) determining the presence or absence of CEMA in the detection zone and optionally determining the presence or absence of Cotinine in the detection zone.
34. Use of the antibody or antigen binding fragment thereof according to any of paragraphs 1 to 13 or the device according to any of paragraphs 25 to 31 for detecting CEMA in a sample.

Any publication cited or described herein provides relevant information disclosed prior to the filing date of the present application. Statements herein are not to be construed as an admission that the inventors are not entitled to antedate such disclosures. All publications mentioned in the above specification are herein incorporated by reference. Various modifications and variations of the invention will be apparent to those skilled in the art without departing from the scope and spirit of the invention. Although the invention has been described in connection with specific preferred embodiments, the invention as claimed should not be unduly limited to such specific embodiments. Indeed, various modifications of the described modes for carrying out the invention which are obvious to those skilled in immunology, cellular or molecular biology or related fields are intended to be within the scope of the following claims.

POLYPEPTIDE SEQUENCES
B11 (VH in bold; VL not bold; CDRs underlined)
SEQ ID NO: 1
QEQLVESGGRLITPGGSLTLTCTVSGIDLNAYGVIWVRQAPGKGLEWIGGISRIGETWYASLVKGRFTISKTSTT
VDLKMTSLTTEDTATYFCARERDAGAVGDGYINDYFNIWGPGTLVTVSELVMTQTPASVSEPVGGTVTINCQASE
TIWSGLAWYQQKPGQPPKLLIYDASNLETGVPSRERGSGSATQFTLTISDLECDDAATYYCQGPYYRSSGYFPFG
GGTELEIL
B11 Heavy chain (VH)
SEQ ID NO: 2
QEQLVESGGRLITPGGSLTLTCTVSGIDLNAYGVIWVRQAPGKGLEWIGGISRIGETWYASLVKGRFTISKTSTT
VDLKMTSLTTEDTATYFCARERDAGAVGDGYTNDYFNIWGPGTLVTVSL
B11 VH CDR1
SEQ ID NO: 3
AYGVI
B11 VH CDR2
SEQ ID NO: 4
GISRIGETWYASLVKG
B11 VH CDR3
SEQ ID NO: 5
ERDAGAVGDGYINDYENI
B11 Light chain (VL)
SEQ ID NO: 6
ELVMTQTPASVSEPVGGTVTINCQASETIWSGLAWYQQKPGQPPKLLIYDASNLETGVPSRERGSGSATQFTLTI
SDLECDDAATYYCQGPYYRSSGYFPFGGGTELEIL
B11 VL CDR1
SEQ ID NO: 7
QASETIWSGLA
B11 VL CDR2
SEQ ID NO: 8
DASNLET
B11 VL CDR3
SEQ ID NO: 9
QGPYYRSSGYFP
All according to Kabat nomenclature
E6 (VH in bold; VL not bold; CDRs underlined)
SEQ ID NO: 10
QSVEESGGRLVTPGTPLTLTCTVSGFSLSSYPVSWVRQAPGKGLEWIGFINTGGSAYYASWAKGRFTISRTSTSV
DLKMASLTASDTATYFCGVNGIWGPGTLVTVSLELDMTQTPPSLSASVGETVRITCLASEDIYSGISWYQQKPGK
PPTLLISGASNLESGVPPRFSGSGSGTDYTITIGGVQAEDAATYYCLGGVSFSTTGTTFGAGTNVEIK
E6 Heavy chain (VH)
SEQ ID NO: 11
QSVEESGGRLVTPGTPLTLTCTVSGFSLSSYPVSWVRQAPGKGLEWIGFINTGGSAYYASWAKGRFTISRTSTSV
DLKMASLTASDTATYFCGVNGIWGPGTLVTVSL
E6 VH CDR1
SEQ ID NO: 12
SYPVS
E6 VH CDR2
SEQ ID NO: 13
FINTGGSAYYASWAKG
E6 VH CDR3
SEQ ID NO: 14
NGI
E6 Light chain (VL)
SEQ ID NO: 15
ELDMTQTPPSLSASVGETVRITCLASEDIYSGISWYQQKPGKPPTLLISGASNLESGVPPRESGSGSGTDYTLTI
GGVQAEDAATYYCLGGVSFSTTGTTFGAGINVEIK
E6 VL CDR1
SEQ ID NO: 16
LASEDIYSGIS
E6 VL CDR2
SEQ ID NO: 17
GASNLES
E6 VL CDR3
SEQ ID NO: 18
LGGVSFSTTGTT
All according to Kabat nomenclature
G4 (VH in bold; VL not bold; CDRs underlined)
SEQ ID NO: 19
QSLEESGGRLVTPGGSLTLTCTVSGIDLNSYGVIWVRQAPGKGLEWIGGISRIGETWYASLVKGRFTISKTSTTV
DLKMTSLTTEDTATYFCARERDAGAVGDGYINDYFNIWGPGTLVIVSLELVMTQTPASVSEPVGGTVTINCQASE
TIWSGLAWYQQKPGQPPKLLIYDASNLETGVPSRFRGSGSATQFTLTISDLECDDAATYYCQGPYYRSSGYFPFG
GGTELEIL
G4 Heavy chain (VH)
SEQ ID NO: 20
QSLEESGGRLVTPGGSLTLTCTVSGIDLNSYGVIWVRQAPGKGLEWIGGISRIGETWYASLVKGRFTISKTSTTV
DLKMTSLTTEDTATYFCARERDAGAVGDGYINDYFNIWGPGTLVIVSL
G4 VH CDR1
SEQ ID NO: 21
SYGVI
G4 VH CDR2
SEQ ID NO: 22
GISRIGETWYASLVKG
G4 VH CDR3
SEQ ID NO: 23
ERDAGAVGDGYTNDYFNI
G4 Light chain (VL)
SEQ ID NO: 24
ELVMTQTPASVSEPVGGTVTINCQASETIWSGLAWYQQKPGQPPKLLIYDASNLETGVPSRFRGSGSATQFTLTI
SDLECDDAATYYCQGPYYRSSGYFPFGGGTELEIL
G4 VL CDR1
SEQ ID NO: 25
QASETIWSGLA
G4 VL CDR2
SEQ ID NO: 26
DASNLET
G4 VL CDR3
SEQ ID NO: 27
QGPYYRSSGYFP
All according to Kabat nomenclature
POLYNUCLEOTIDE SEQUENCES
B11 (VH in bold; VL not bold)
SEQ ID NO: 28
Caggagcaattggtggaatcaggcgggagactgattaccccaggtggaagccttacactgacttgcactgtctca
gggattgacctgaatgcctatggcgtcatctgggttcgccaagcaccagggaaagggctggagtggattggaggc
ataagtcggattggggaaacctggtatgccagtctggtgaaaggtcgctttactatctccaagacatccaccaca
gtggacctcaagatgacctctctgactacggaggacacagctacctatttctgtgcacgagagagggatgctgga
gctgttggtgatgggtacaccaacgactacttcaacatctggggacccggcactcttgtgaccgtgagcctggag
ctggtcatgacacagacccctgctagcgtgtcagaaccagtgggtggtacagtcaccatcaattgccaggcttcc
gagacaatatggtccggattggcctggtatcagcagaagcctggacagccacccaaactcctgatctacgatgcc
tccaacctggaaacaggcgtacctagccgttttcgaggcagtggctctgcaactcagttcacccttacgatcagc
gacttggaatgcgatgatgccgcaacctactactgtcaagggccctattatcggtctagtggatactttcccttt
ggcggcggcactgagctggagattctc
B11 Heavy chain (VH)
SEQ ID NO: 29
Caggagcaattggtggaatcaggcgggagactgattaccccaggtggaagccttacactgacttgcactgtctca
gggattgacctgaatgcctatggcgtcatctgggttcgccaagcaccagggaaagggctggagtggattggaggc
ataagtcggattggggaaacctggtatgccagtctggtgaaaggtcgctttactatctccaagacatccaccaca
gtggacctcaagatgacctctctgactacggaggacacagctacctatttctgtgcacgagagagggatgctgga
gctgttggtgatgggtacaccaacgactacttcaacatctggggacccggcactcttgtgaccgtgagcctg
B11 Light chain (VL)
SEQ ID NO: 30
gagctggtcatgacacagacccctgctagcgtgtcagaaccagtgtggggtacagtcaccatcaattgccaggct
tccgagacaatatggtccggattggcctggtatcagcagaagcctggacagccacccaaactcctgatctacgat
gcctccaacctggaaacaggcgtacctagccgttttcgaggcagtggctctgcaactcagttcacccttacgatc
agcgacttggaatgcgatgatgccgcaacctactactgtcaagggccctattatcggtctagtggatactttccc
tttggcggggcactgagctggagattctc
E6 (VH in bold; VL not bold)
SEQ ID NO: 31
cagtcggtggaggagtccgggggtcgcctggtcacgcctgggacacccctgacactcacctgcacagtctctgga
ttctccctcagtagctatccagtgagctgggtccgccaggctccagggaaggggctggaatggatcggatttatt
aatactggtggtagcgcatactacgcgagctgggcaaaaggccgattcaccatctccagaacctcgacctcggtg
gatctgaaaatggccagtctgacagcctcggacacggccacctatttctgtggtgtaaatggtatctggggccca
ggcaccctggtcaccgtctccttagagctcgatatgacccagactccaccctccctgtctgcatctgtgggagaa
actgtcaggattacgtgcctggccagtgaggacatttacagtggtatatcctggtatcaacagaagccagggaaa
cctcctacactcctgatctctggtgcatccaatttagaatctggggtcccaccacggttcagtggcagtggatct
gggacagattacaccctcaccattggcggcgtgcaggctgaagatgctgccacctactactgtctaggcggtgtt
agtttcagtactaccggtacgacttttggagctggcaccaatgtggaaatcaaa
E6 Heavy chain (VH)
SEQ ID NO: 32
Cagtcggtggaggagtccgggggtcgcctggtcacgcctgggacacccctgacactcacctgcacagtctctgga
ttctccctcagtagctatccagtgagctgggtccgccaggctccagggaaggggctggaatggatcggatttatt
aatactggtggtagcgcatactacgcgagctgggcaaaaggccgattcaccatctccagaacctcgacctcggtg
gatctgaaaatggccagtctgacagcctcggacacggccacctatttctgtggtgtaaatggtatctggggccca
ggcaccctggtcaccgtctcctta
E6 Light chain (VL)
SEQ ID NO: 33
gagctcgatatgacccagactccaccctccctgtctgcatctgtgggagaaactgtcaggattacgtgcctggcc
agtgaggacatttacagtggtatatcctggtatcaacagaagccagggaaacctcctacactcctgatctctggt
gcatccaatttagaatctggggtcccaccacggttcagtggcagtggatctgggacagattacaccctcaccatt
ggcggcgtgcaggctgaagatgctgccacctactactgtctaggcggtgttagtttcagtactaccggtacgact
tttggagctggcaccaatgtggaaatcaaac
G4 (VH in bold; VL not bold)
SEQ ID NO: 34
cagtcgctggaggagtccgggggtcgcctggtaacgcctggaggatccctgacactcacctgcacagtctctgga
atcgacctcaattcttatggagtgatctgggtccgccaggctccagggaaggggctggaatggatcggaggcatc
agtaggattggtgagacatggtacgcgagcttggtgaaaggccgattcaccatctccaaaacctcgaccacggtg
gatctgaaaatgaccagtctgacaaccgaggacacggccacctatttctgtgccagagaacgtgatgctggtgcg
gttggtgatggttatactaacgactactttaatatctggggcccaggcaccctggtcatcgtctccttagagctc
gtgatgacccagactccagcctccgtgtctgaacctgtgggaggcacagtcaccatcaattgccaggccagtgag
actatttggagtggtttggcctggtatcagcagaaaccagggcagcctcccaaactcctgatctatgatgcatcc
aatctggagactggggtcccatcgcggttcagaggcagtggatctgcgacacagttcactctcaccatcagcgac
ctggagtgtgacgatgctgccacttactattgtcaaggtccttattataggagtagtggctattttcctttcggc
ggagggaccgagctggagatccta
G4 Heavy chain (VH)
SEQ ID NO: 35
cagtcgctggaggagtccgggggtcgcctggtaacgcctggaggatccctgacactcacctgcacagtctctgga
atcgacctcaattcttatggagtgatctgggtccgccaggctccagggaaggggctggaatggatcggaggcatc
agtaggattggtgagacatggtacgcgagcttggtgaaaggccgattcaccatctccaaaacctcgaccacggtg
gatctgaaaatgaccagtctgacaaccgaggacacggccacctatttctgtgccagagaacgtgatgctggtgcg
gttggtgatggttatactaacgactactttaatatctggggcccaggcaccctggtcatcgtctcctta
G4 Light chain (VL)
SEQ ID NO: 36
gagctcgtgatgacccagactccagcctccgtgtctgaacctgtgggaggcacagtcaccatcaattgccaggcc
agtgagactatttggagtggtttggcctggtatcagcagaaaccagggcagcctoccaaactcctgatctatgat
gcatccaatctggagactggggtcccatcgcggttcagaggcagtggatctgcgacacagttcactctcaccatc
agcgacctggagtgtgacgatgetgccacttactattgtcaaggtccttattataggagtagtggctattttcct
ttcggcggagggaccgagctggagatccta

TABLE 1
Amino acid sequence analysis of B11, E6 and
G4 scFv according to Kabat numbering

| VH | | | | VL | | | |
|---|---|---|---|---|---|---|---|
| | Sequence | | | | Sequence | | |
| Region | Fragment | Residues | Length | Region | Fragment | Residues | Length |

5255-137-R4C-B11

| | | | | | | | |
|---|---|---|---|---|---|---|---|
| HFR1 | QFQLVESGGRLITPGG | 01-30 | 30 | LFR1 | | 01-23 | 23 |
| | SLTLTCTVSGIQLN | | | | | | |
| CDR-H1 | AYGVI | 31-35 | 5 | CDR-L1 | QASETIVSGLA | 24-34 | 13 |
| HFR2 | WVRQAPGKGLEWIG | 36-49 | 14 | LFR2 | WYQQKPGQPPKLUY | 35-49 | 15 |
| CDR-H2 | GISRIGETWYASLVKG | 50-65 | 16 | CDR-L2 | DASNLET | 50-56 | 7 |
| HFR3 | RFTISKTSTTVDLKMT | 66-95 | 30 | LFR3 | GYPSYFAGSGSATQ | 57-88 | 32 |
| | SLTTEDTATYFCAR | | | | FTLTISDLECDDAA | | |
| | | | | | TYYC | | |
| CDR-H3 | ERDAGAVGDGYTNDYF | 96-113 | 18 | CDR-L3 | QGPYYRSSGYES | 89-100 | 12 |
| | NI | | | | | | |
| HFR4 | WGPSTLVTVSL | 114-124 | 11 | LFR4 | FGGGTELEIL | 101-110 | 10 |

5255-137-REC-E6

| | | | | | | | |
|---|---|---|---|---|---|---|---|
| HFR1 | QSVEESGGRLVTPGTP | 1-29 | 29 | LFR1 | ELQMTQTPPSLSAS | 01-23 | 23 |
| | LTLTCTYSGFSLS | | | | YGETVRITC | | |
| CDR-H1 | SYFVS | 30-34 | 5 | CDR-L1 | LASEDIYEGIS | 24-34 | 12 |
| HFR2 | WYRQAPGKGLEWIG | 35-48 | 14 | LFR2 | WYQQXPGKPPTLLI | 35-49 | 15 |
| | | | | | S | | |
| CDR-H2 | ENTGGSAYYASWAKGR | 49-64 | 15 | CDR-L2 | GASNLES | 50-55 | 7 |
| HFR3 | FTISRTSTSVQLKMAS | 65-94 | 30 | LFR3 | GMPPRESGEGSGTQ | 57-88 | |
| | LTASDTATYFCGV | | | | YTLTIGGVQAEDAA | | |
| | | | | | TYYC | | |
| CDR-H3 | NGI | 95-97 | 3 | CDR-L3 | LGGVSFSTTGTT | 89-100 | 12 |
| HFR4 | WGPGTLVTVSL | 98-108 | 11 | LFR4 | FGASTNVBX | 101-110 | 10 |

5255-137-R5C-G4

| | | | | | | | |
|---|---|---|---|---|---|---|---|
| HFR1 | QSLEESGGRLVTPGGS | 1-29 | 29 | LFR1 | ELVMTQTPASVSEF | 01-23 | 23 |
| | LTLTCTVSGIDLN | | | | VGGTVTINC | | |
| CDR-H1 | SYGW | 30-34 | 5 | CDR-L1 | DASETISGLA | 24-34 | 15 |
| HFR2 | WVRDAPGKGLEWIG | 35-48 | 14 | LFR2 | WYQQKPGQPPKLUY | 35-49 | 15 |
| CDR-H2 | GISRIGETWYASLVKG | 49-64 | 15 | CDR-L2 | DASNLET | 50-55 | 7 |
| HFR3 | RFTISKTSTTVDLKMT | 65-94 | 30 | LFR3 | GPSEFRGSGGATQF | 57-88 | 32 |
| | SLTTEDTATYFCAR | | | | TLTISDLECDDAAT | | |
| | | | | | YYC | | |
| CDR-H3 | ERDAGAVGDGYTNDYF | 95-122 | 18 | CDR-L3 | QGPYYRSSGYES | 89-100 | 12 |
| | NI | | | | | | |
| HFR4 | WGPGTLVIVSL | 113-123 | 13 | LFR4 | FGGGTELEIL | 101-110 | 10 |

1.-15. (canceled)

16. An antibody or antigen binding fragment thereof comprising:
VH CDR1 consisting of the amino acid sequence of SEQ ID NO: 3, a VH CDR2 consisting of the amino acid sequence of SEQ ID NO: 4 and a VH CDR3 consisting of the amino acid sequence of SEQ ID NO: 5, and a VL CDR1 consisting of the amino acid sequence of SEQ ID NO: 7, a VL CDR2 consisting of the amino acid sequence of SEQ ID NO: 8 and a VL CDR3 consisting of the amino acid sequence of SEQ ID NO: 9; or
VH CDR1 consisting of the amino acid sequence SEQ ID NO: 12, a VH CDR2 consisting of the amino acid sequence SEQ ID NO:13 and a VH CDR3 consisting of the amino acid sequence SEQ ID NO: 14, and a VL CDR1 consisting of the amino acid sequence SEQ ID NO:16, a VL CDR2 consisting of the amino acid sequence SEQ ID NO: 17 and a VL CDR3 consisting of the amino acid sequence SEQ ID NO: 18; or
VH CDR1 consisting of the amino acid sequence SEQ ID NO:21, a VH CDR2 consisting of the amino acid sequence SEQ ID NO:22 and a VH CDR3 consisting of the amino acid sequence SEQ ID NO: 23, and a VL CDR1 consisting of the amino acid sequence SEQ ID NO: 25, a VL CDR2 consisting of the amino acid sequence SEQ ID NO:26 and a VL CDR3 consisting of the amino acid sequence of SEQ ID NO: 27.

17. The antibody according to claim 16, wherein the antibody or antigen binding fragment thereof comprises a VH amino acid sequence consisting of the amino acid sequence of SEQ ID NO: 2, 11, or 20; and/or
wherein the antibody or antigen binding fragment thereof comprises a VL amino acid sequence consisting of the amino acid sequence of SEQ ID NO: 6, 15, or 24.

18. The antibody according to claim 16, wherein the antibody or antigen binding fragment thereof comprises VH amino acid sequence consisting of the amino acid sequence of SEQ ID NO: 2 and VL amino acid sequence consisting of the amino acid sequence of SEQ ID NO:6; or
wherein said antibody or antigen binding fragment thereof comprises VH amino acid sequence consisting of the amino acid sequence of SEQ ID NO:11 and VL amino acid sequence consisting of the amino acid sequence of SEQ ID NO:15; or
wherein said antibody or antigen binding fragment thereof comprises VH amino acid sequence consisting of the amino acid sequence of SEQ ID NO: 20 and VL amino acid sequence consisting of the amino acid sequence of SEQ ID NO: 24.

19. The antibody or antigen binding fragment thereof according to claim 16,
wherein the antigen binding fragment thereof is selected from the group consisting of a Fab fragment, a Fab′ fragment, a F(ab′)₂ fragment, a scFv, a Fv, a rIgG, and a diabody,
wherein the antigen binding fragment is a scFv and
wherein said scFv comprises the amino acid sequence of SEQ ID NO: 1 or SEQ ID NO: 10 or SEQ ID NO: 19.

20. The antibody or antigen binding fragment thereof according to claim 16, wherein the antibody does not bind to cotinine or 2-hydroxyethylmethacrylate (HEMA) or monohydroxybutenyl-mercapturic acid (MHBMA) or 3-hydroxypropylmercapturic acid (3-HPMA) or dihydroxybutyl mercapturic acid (DHBMA).

21. A complex of the antibody or antigen binding fragment thereof according to claim 16 and a compound of Formula [I] or Formula [II].

22. A complex of the antibody or antigen binding fragment thereof according to claim 16 immobilized on a solid phase.

23. A complex of the antibody or antigen binding fragment thereof according to claim 16 and a pad, dipstick or other lateral flow device.

24. A complex of the antibody or antigen binding fragment thereof according to claim 16 and a detectable label.

25. A device for determining the presence or absence of cyanoethyl mercapturic acid (CEMA) in a sample, said device comprising the antibody or antigen binding fragment thereof according to claim 16 immobilised on a solid phase of the device.

26. The device according to claim 25 that comprises a portable lateral flow immunoassay device or a dipstick,
wherein the device comprises:
(i) a sample pad for receiving a sample;
(ii) a conjugate pad in fluid communication with the sample pad;
(iii) at least one detection zone in fluid communication with a distal end of the conjugate pad; and
(iv) an adsorbent pad in fluid communication with a distal end of the detection zone.

27. The device according to claim 26,
wherein the conjugate pad comprises the antibody or antigen binding fragment thereof in a labelled form; or
wherein the conjugate pad comprises the antibody or antigen binding fragment thereof in a labelled form and further comprises a labelled antibody or antigen binding fragment thereof that binds to cotinine; or
wherein the antibody or antigen binding fragment thereof is labelled and is separately contained in an intermediate pad of the device that is located adjacent the conjugate pad; or
wherein the antibody or antigen binding fragment thereof is labelled and is separately contained in an intermediate pad of the device that is located adjacent the conjugate pad and wherein a labelled antibody or antigen binding fragment thereof capable of binding cotinine is separately contained in the conjugate pad.

28. The device according to claim 25, wherein the detection zone comprises CEMA and/or cotinine immobilised thereon.

29. The device according to claim 28, wherein the CEMA is in the form of a conjugate comprising a compound of formula [II]: 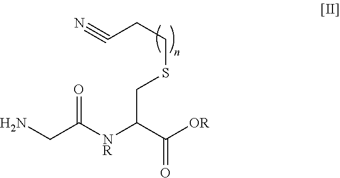
wherein n is selected from 0, 1, 2, 3 or 4 and each R is independently selected from H or C₁ to C₆ alkyl; or
a compound of formula [I]: 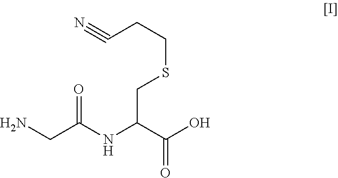 [SMILES: N#CCCSCC(NC(=O)CN)C(=O)O]
wherein the compound of formula [II] or formula [I] is coupled to an immunogenic carrier via a linker, and wherein the linker is coupled to the compound of formula [I] via the amine group.

30. The device according to claim 29, wherein the immunogenic carrier is bovine serum albumin, bovine thyroglobulin, or another carrier protein; and/or wherein the linker is glycol bis(succinimidyl succinate) (EGS) or disuccinimidyl suberate (DSS).

31. A method for detecting CEMA, or CEMA and cotinine, in a sample comprising contacting the sample with the antibody or antigen binding fragment thereof which has been immobilized on a solid phase of the device according to claim 25.

32. A method for determining smoking status of a subject by detecting cyanoethyl mercapturic acid (CEMA) in a sample comprising:
contacting the sample with the antibody or antigen binding fragment thereof according to claim 16,
detecting binding of the sample to the antibody or antigen binding fragment thereof, and
determining smoking status based on an amount of binding detected compared to a control value.

33. The method of claim 32, wherein the sample comprises urine.

34. A method for producing an antibody that recognizes cyanoethyl mercapturic acid (CEMA) comprising inducing an immune response to a conjugate of CEMA and an immunogenic carrier or to CEMA conjugated via a linker to the immunogenic carrier

35. The method of claim 34, further comprising selecting an antibody that binds to CEMA but that does not bind to cotinine or 2-hydroxyethylmethacrylate (HEMA) or monohydroxybutenyl-mercapturic acid (MHBMA) or 3-hydroxypropylmercapturic acid (3-HPMA) or dihydroxybutyl mercapturic acid (DHBMA).
